(19) United States
(12) Patent Application Publication
(10) Pub. No.: US 2025/0031941 A1
(43) Pub. Date: Jan. 30, 2025

(54) MEDICAL PROCEDURE PREPARATION SYSTEM, AND MEDICAL PROCEDURE PREPARATION METHOD

(71) Applicant: OLYMPUS MEDICAL SYSTEMS CORP., Tokyo (JP)

(72) Inventors: Junya YAMADA, Tokyo (JP); Kazuma KANEKO, Tokyo (JP); Osamu NONAKA, Sagamihara-shi (JP); Hisato KOGURE, Hidaka-shi (JP); Manabu ICHIKAWA, Tokyo (JP); Kento IGUCHI, Tokyo (JP); Natsuko ODAKA, Tokyo (JP)

(73) Assignee: OLYMPUS MEDICAL SYSTEMS CORP., Tokyo (JP)

(21) Appl. No.: 18/782,254

(22) Filed: Jul. 24, 2024

Related U.S. Application Data
(60) Provisional application No. 63/528,932, filed on Jul. 26, 2023.

(51) Int. Cl. A61B 1/00; G16H 10/60; G16H 40/40

(52) U.S. Cl. CPC A61B 1/00006; A61B 1/00039; A61B 1/00045; G16H 10/60; G16H 40/40

(57) ABSTRACT

A medical procedure preparation system includes an input interface, a display, at least one a processor. The at least one processor is configured to receive an input containing (i) a medical procedure location where a medical procedure is performed, and (ii) a storage location where a medical device is stored, calculate (iii) a distance between (i) and (ii), calculate one of (a) a preparation start time of the medical device used in the medical procedure and (b) a performing time of the medical procedure using the medical device based on (iii) and (iv), and output a display image indicating one of (a) and (b) to the display. Calculation one of (a) and (b) is based on (iii) the distance and (iv) a movement speed of a transport device.

### RELATED APPLICATION DATA

This application is based on and claims priority under 35 U.S.C. § 119 to U.S. Provisional Application No. 63/528,932, filed on Jul. 26, 2023, the entire contents of which are incorporated herein by reference.

The present disclosure relates to a medical procedure preparation apparatus, a medical procedure preparation method, a medical system, an endoscopy preparation apparatus, and a non-transitory recording medium recording a medical procedure preparation program that facilitate preparation for a medical procedure.

### BACKGROUND

Medical devices used in various medical procedures in medical institutions need to be cleaned as necessary, and also, a certain space is required to correctly manage a number of various types of medical devices and to store the medical devices so as to be easily taken out when necessary. Accordingly, a medical device is generally stored or kept until needed at a location different from a location where a medical procedure is performed, such as an examination room, and is carried into the examination room or the like from a storage/standby location at the time of examination, for example.

On the other hand, a patient who is to receive a medical procedure such as an examination (a healthy examination subject may be referred to as an examinee, but in the following description, a healthy examination subject may be referred to as a patient), needs to be prepared for the examination, preparations including schedule management according to each patient's life, health management for bringing the patient into good condition for the examination, preliminary examination, dietary restriction, and the like. In other words, before a medical procedure such as an examination, various actions for schedule management/adjustment need to be performed in relation to medical workers and facilities. Besides, medical devices need to be carried to the examination room or the like, and prepared for use. The patient needs to prepare for the examination or the like. Accordingly, before performing a medical procedure, strict schedule management is necessary.

### SUMMARY

A medical procedure preparation system includes an input interface, a display, at least one processor comprising hardware. The at least one processor configured to receive an input containing (i) a medical procedure location where a medical procedure is performed, and (ii) a storage location where a medical device is stored, calculate (iii) a distance between (i) the medical procedure location and (ii) the storage location, calculate one of (a) a preparation start time of the medical device used in the medical procedure and (b) a performing time of the medical procedure using the medical device, and output a display image indicating one of (a) and (b) to the display. Calculation one of (a) and (b) is based on (iii) the distance and (iv) a movement speed of a transport device.

A medical procedure preparation method comprises receiving an input containing (i) a medical procedure location where a medical procedure is performed, and (ii) a storage location where a medical device is stored from an input interface, calculating (iii) a distance between (i) the medical procedure location and (ii) the storage location where the medical device is stored, and calculating one of (a) a preparation start time of the medical device used in the medical procedure and (b) a performing time of the medical procedure using the medical device, and outputting a display image indicating one of (a) and (b) to a display. Calculation one of (a) and (b) is based on (iii) the distance and (iv) a movement speed of a transport device.

A medical procedure preparation method, comprises receiving an input signal from an input interface, obtaining, based on the input signal, a medical procedure location where the medical procedure is performed, a storage location where a medical device is stored, a performing time of the medical procedure using the medical device, calculating one of a preparation start time of the medical device, and outputting the preparation start time. The medical device is used in the medical procedure

### BRIEF DESCRIPTION OF THE DRAWINGS

FIG. 1 is a block diagram showing a medical procedure preparation apparatus according to a first embodiment of the present disclosure.

FIG. 2 is an explanatory diagram for describing information about an arrangement at the time of endoscopy.

FIG. 3 is an explanatory diagram for describing preparation for performing a medical procedure.

FIG. 4 is a flowchart for describing an operation according to the first embodiment.

FIG. 5 is an explanatory diagram for describing a schedule of a medical procedure.

FIG. 6 is an explanatory diagram showing an example of a location database.

FIG. 7 is an explanatory diagram showing an example display of a schedule display including a check function.

FIG. 8 is an explanatory diagram showing a preparation guide display.

FIG. 9 is a block diagram showing a transport device 50A adopted in a modification.

FIG. 10 is a flowchart for describing an operation after start of endoscopy.

FIG. 11 is a flowchart showing an operation flow adopted in a second embodiment.

FIG. 12 is an explanatory diagram showing an example of a transport route of a medical device.

FIG. 13 is an explanatory diagram for describing an operation according to the second embodiment.

FIG. 14 is a flowchart showing an operation flow adopted in a third embodiment.

FIG. 15 is an explanatory diagram showing an example of a transport route of a medical device according to the third embodiment.

### DETAILED DESCRIPTION

Hereinafter, embodiments of the present disclosure will be described in detail with reference to the drawings.

### First Embodiment

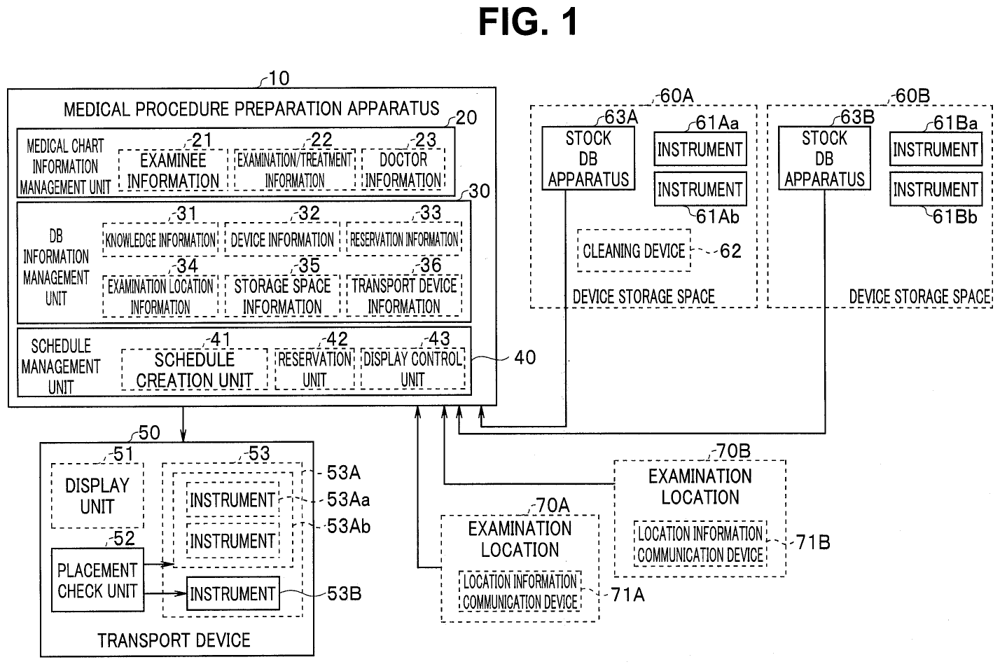

FIG. 1 is a block diagram of a medical system including a medical procedure preparation apparatus according to a first embodiment of the present disclosure. The present embodiment effectively supports schedule management for performing a medical procedure, based on a positional relationship between a service provision location of the medical procedure and a storage space for a medical device. Note that in the present specification, a medical device refers to a collection of one or more devices for performing a predetermined medical procedure. In the present embodiment, an example is described where a medical device including an endoscope is used as the medical device and preparation for endoscopy is supported. However, the medical device need not include an endoscope, and is also applicable to any apparatus that supports preparation of various medical procedures including various examinations and various treatments without being limited to endoscopy, such that a treatment instrument. Note that, in the endoscopy, only examination may be performed or the examination may be performed on the premise that a treatment will also be performed, and thus in the following description, the simple term “examination” may include a treatment.

Reference is made to medical procedures herein, but the preparation apparatus can be applied to a technology where a target object to be examined using an apparatus may not be a target object of a medical procedure, and thus, is applicable to an apparatus that supports preparation of various examination procedures including various examinations and various treatments.

Conventional medical services are generally provided when a person visits a medical institution such as a hospital as the person feels anxious about his/her health or for a medical checkup. Nowadays, there is no restriction regarding a place where patients receive a medical service owing to an increase in information processing capacity or improvement in communication environment, and patients are allowed to select a place for receiving a service.

Furthermore, in relation to a problem of concentration of patients in large hospitals, nowadays, individual clinics and the like serve the role of “family doctor”, and some examinations which had been performed in large hospitals are being performed in a family medical institution.

Such a clinic or the like often does not have a dedicated space for storing medical devices, and a medical device may be carried from a station at another location to the clinic or the like where a service of a medical procedure is provided (hereinafter also referred to as a medical procedure performing location).

Furthermore, a place for another purpose may be used as a temporary storage location, and taking out the medical device may be burdensome. Moreover, even in large hospitals, a storage location of the medical device and the medical procedure performing location are often different. For example, since some medical devices, such as an endoscope, need cleaning, a medical device to be used may be selected from among many devices depending on the type of examination, a relatively large storage space is needed, and so on, medical devices are often stored in a specific storage location.

Accordingly, in the case where an examinee is to be subjected to an examination or a treatment in an examination room or the like in a large hospital, the medical device may need to be carried from the storage space. Furthermore, a transport device, such as a cart, for carrying the medical device may also be kept at a different location. Moreover, a simple examination may be performed not in the examination room but in a patient room, and the medical device need to be carried at the time of the medical procedure. Of course, a device that has just been purchased or rented and carried from a purchase source or a rental source may be used. In such a case, a package reception room or the like in the hospital may be considered a standby location or a storage location, or depending on a situation, the present application may be applied in relation to movement from a delivery source.

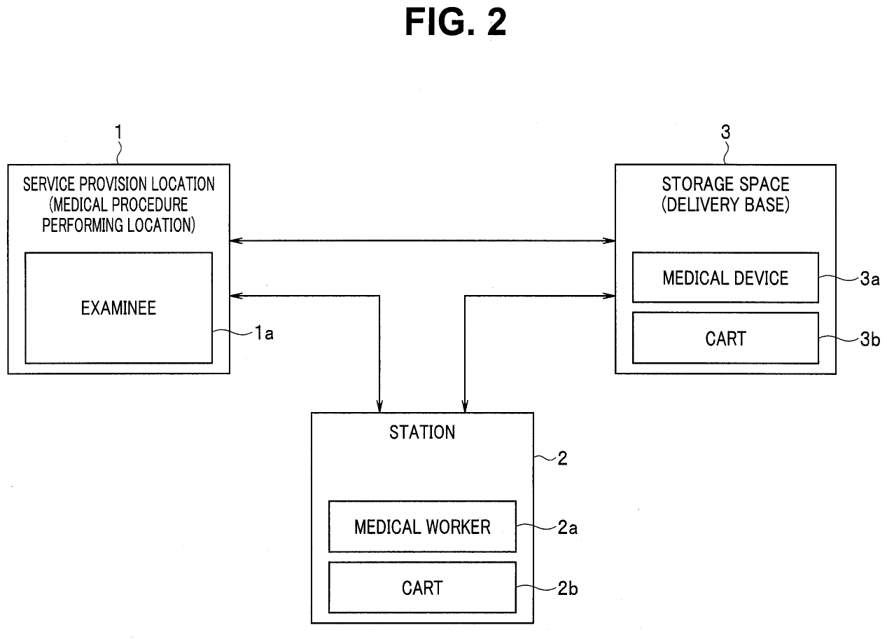

FIG. 2 is an explanatory diagram for describing a relationship among a service provision location 1 as a medical procedure performing location where an examination or a treatment is performed, a staff station (hereinafter “station”) 2 where medical workers are stationed, and a storage space 3.

The service provision location 1 is a location where a medical procedure is performed, such as an examination room. A medical device to be used in the medical procedure at the service provision location 1 is stored in the storage space 3, such as a delivery base. A cart 2b for carrying the medical device to an examinee is located at the station 2 where medical workers are stationed.

An examinee 1a who is ready to receive a medical procedure waits at the service provision location 1. A medical worker 2a stationed at the station 2 moves to and from between the service provision location 1 and the storage space 3. The medical worker 2a carries the cart 2b to the storage space 3, places a medical device 3a stored in the storage space 3 on the cart 2b, and then, heads to the service provision location 1. Note that a cart 3b may be provided in the storage space 3. A doctor, who is a medical worker, may thus perform a medical procedure on the examinee at the service provision location 1 using the medical device 3a.

In other words, to perform a medical procedure, not only preparation on the side of a patient, but also preparation of the medical device is necessary, and consideration must be given to the time period to prepare the medical device. Accordingly, to efficiently perform the medical procedure without placing a burden on the patient, scheduling including reservation to use the medical device is important. A burden on the patient may be physical or mental stress caused by being tied down by time due to a waiting time for an examination, an examination time, a medical procedure related to an examination, paperwork, and the like. Of course, an examination may not be performed smoothly and the patient may need to be re-examined or need to visit the hospital again. Considerations must be given to financial burden of a patient, influence on daily life, body and mind of an attending person including financial and temporal burden, and the like.

Note that Japanese Patent Application Laid-Open Publication No. 2023-32596 discloses a technology of obtaining surgical procedure information, information about a surgical instrument used in each procedure, and individual information (i.e. medical chart information) of a patient, estimating uncertainties regarding the patient (unexpectedly treatments), creating an individual surgery model associating a surgical instrument used to the treatment with the patient, and outputting information about a candidate surgical instrument. However, the proposed technology does not give consideration to the time period to prepare a medical device, and does not appropriately and reliably support scheduling of a medical procedure, and does not always efficiently support a medical procedure including a preparation stage and a schedule adjustment.

A medical system in FIG. 1 includes a medical procedure preparation apparatus 10 configured to perform scheduling for a medical procedure based on a positional relationship among a transport device 50, device storage spaces 60A, 60B, and examination locations 70A, 70B.

The transport device 50 is a device for carrying the medical device, such as a push cart or a self-propelled robot. For example, the transport device 50 corresponds to the carts 2b, 3b in FIG. 2. Depending on conditions such as a distance by which the medical device is to be carried, a vehicle such as a car may be adopted as the transport device 50. The transport device 50 may include a display unit (display) 51. An instrument 53, which is a medical device, may be placed on the transport device 50. FIG. 1 indicates that an instrument 53B is placed on the transport device 50, but various medical devices including instruments 53Aa, 53Ab indicated by dashed lines may be placed on the transport device 50. Note that the instrument 53Ab is a backup medical device for the instrument 53Aa. Note that after a medical device is used, the medical device must be correctly disposed of according to a predetermined procedure or must be collected to be reused, and the transport device 50 may be used for such disposal or collection. The transport device 50 is configured to carry the medical device from (ii) the storage location to (i) the medical procedure location.

A placement check unit 52 holds information about the medical device that is placed on the transport device 50. The placement check unit 52 may be configured with at least one processor that uses a central processing unit (CPU) and the like. The placement check unit 52 may operate according to a program stored in a memory, not shown to control each unit, or some or all the functions may be implemented by an electronic circuit of hardware, by installed application or by installed program. The placement check unit 52 can obtain information about a medical device to be used in a medical procedure, by exchanging information with a schedule management unit 40 described later.

Furthermore, the placement check unit 52 may obtain information about the medical device placed on the transport device 50 from a camera configured to capture the instrument 53 or from a communication device configured to read information in an IC tag or the like provided on the instrument 53. Moreover, the placement check unit 52 may obtain information about the medical device placed on the transport device 50 through an input operation on an input operation section (input interface) such as a keyboard, a touch screen, a switch, a button, not shown.

The placement check unit 52 generates display data for displaying information about a medical device used for a medical procedure or a medical device placed on the transport device 50, and outputs the display data to the display unit 51. The display unit 51 may be configured with a display device such as an LCD, and displays a display based on the display data from the placement check unit 52 on a display screen. For example, the display unit 51 can display a device (medical device) used for a next examination/treatment, or display whether the device is placed on the transport device 50. Such a display facilitates checking of a medical device that a medical worker should prepare and a medical device that is already prepared, and a preparation task of the medical worker may be effectively supported. Note that the display unit 51 and the placement check unit 52 may be provided at a location other than the transport device 50. For example, the display unit 51 may be provided at any location as long as the display unit 51 can be viewed by the medical worker, and may be configured with a mobile terminal, such as a smartphone, carried by the medical worker.

The device storage spaces 60A, 60B (hereinafter, collectively referred to as device storage space 60 when need not to be distinguished) are storage spaces for storing various medical devices, and correspond to the storage space 3 in FIG. 2. In the example in FIG. 1, instruments 61Aa, 61Ab (hereinafter, collectively referred to as instrument 61A when not needed to be distinguished), which are medical devices, are stored in the device storage space 60A. Furthermore, a cleaning device 62 may be provided in the device storage space 60A. Instruments 61Ba, 61Bb (hereinafter, collectively referred to as instrument 61B when not needed to be distinguished), which are medical devices, are stored in the device storage space 60B. Stock database (DB) apparatuses 63A, 63B are provided in the device storage spaces 60A, 60B, respectively. Note that the device storage space 60A may store medical devices that are relatively frequently used, and the device storage space 60B may store medical devices that are not used often, for example.

The stock DB apparatus 63A, 63B may be configured with at least one processor that uses a CPU and the like, and may operate according to a program stored in a memory, not shown. The stock DB apparatus 63A, 63B includes a recording medium, not shown, and creates and holds a database for managing stock. The stock DB apparatus 63A holds information about the instrument 61A stored in the device storage space 60A, in the form of a database. The stock DB apparatus 63B holds information about the instrument 61B stored in the device storage space 60B, in the form of a database. Note that the stock DB apparatuses 63A, 63B may be provided at locations other than the device storage spaces 60A, 60B.

The examination locations 70A, 70B is a medical procedure performing location where a medical procedure, such as an examination or a treatment, is performed using various medical devices, and corresponds to the service provision location 1 in FIG. 2. Location information communication devices 71A, 71B are provided in the examination locations 70A, 70B, respectively.

The location information communication devices 71A, 71B hold information about the examination locations 70A, 70B (hereinafter, referred to as medical procedure performing location information, examination location information, or medical procedure location) respectively, and can transmit the examination location information to the medical procedure preparation apparatus 10.

Note that the medical procedure performing location may be changed depending on the type of the medical procedure, request from the examinee, or the like, but the location of the station 2 where the transport device 50 is located and the locations of the device storage spaces 60A, 60B where the medical devices are stored (hereinafter, storage location) are assumed to be known. Information about locations of the transport device 50 and the device storage spaces 60A, 60B, that is, transport device location information and storage location information, are assumed to be stored in the medical procedure preparation apparatus 10. Furthermore, a position measuring device such as a global positioning system (GPS) may be provided in the transport device 50, and the transport device location information based on a current position of the transport device 50 may be transmitted to the medical procedure preparation apparatus 10.

Note that “storage location” may imply a location where a target object, such as a medical device, is contained for a specific period of time, but in the present disclosure, the storage location also refers to a location where an object contained or stored is taken out and kept until a medical procedure is actually performed. This is because, although in many facilities, a storage location serves also as a preparation location, in some facilities, a medical device is taken out of the storage space in advance and is placed in a standby location, and such a case may also be considered short-term storage.

### (Medical Procedure Preparation Apparatus)

The medical procedure preparation apparatus 10 includes a medical chart information management unit 20, a database (DB) information management unit 30, and a schedule management unit 40. The medical procedure preparation apparatus 10 may be arranged at any location as long as communication with the transport device 50, the examination location 70, and the like is possible, and may be provided on a server on a cloud, for example, and functions of the medical procedure preparation apparatus 10 may be usable as a Web application on the server.

The medical chart information management unit 20 includes an examinee information memory 21, an examination/treatment information memory 22, and a doctor information memory 23. The medical chart information management unit 20 includes a communication device, not shown, and is capable of obtaining, from a server of an in-hospital system, or cloud computing, not shown, examinee (patient) information, medical chart information such as examination/treatment information, and doctor information about a doctor. The medical chart information management unit 20 stores the examinee information, the examination/treatment information, and the doctor information that are obtained, in the examinee information memory 21, the examination/treatment information memory 22, and the doctor information memory 23, respectively, in the form of databases. Note that the medical chart information management unit 20 may obtain the medical chart information and the doctor information by a user operation on an input operation section (input interface) such as a keyboard, a touch screen, a switch, a button, not shown.

The examinee information includes information for managing physical condition and medical history of each examinee. The examinee information may further include profile information of the examinee. The examination/treatment information includes information about an examination or a treatment, such as an intestinal cleansing examination. In other words, before endoscopy, an examinee needs to be prepared by cleaning an examination part. Preparation often takes time. For example, the examinee starts fasting from previous day or starts taking a medicine for a pretreatment, such as intestinal cleansing, half a day in advance. If the preparation is not correctly performed, then the examination or the treatment will not be correctly performed.

Accordingly, it is important to create a schedule, and to inform the examinee and the medical worker of the schedule. Such an event may be said to be calculation or determination of a preparation start time in a broad sense. Such examination preparation time information about an examinee may be included in the examinee information. In the case where procedures other than standard examination and treatment are necessary for the examinee, the examination/treatment information may be customized accordingly. The medical chart information and the doctor information include information about an examination or a treatment that is to be performed and information about a doctor who is to perform the examination or the treatment. The medical chart information is created by a doctor. For example, a doctor creates the medical chart information by examining the examinee.

The at least one processor is further configured to receive the medical chart information, and determine the medical device used in the medical procedure based on the medical procedure. The medical chart information can include a type of the medical procedure to be performed on the examinee.

The at least one processor can be configured to generate a schedule of the medical procedure including (a) the preparation start time and (b) the performing time, and output the schedule to at least one of the examinee or the medical professional. Generating the schedule can be based on one or more of (i) the medical procedure location, (ii) the storage location, (iv) the movement speed of the transport device 50, (v) the location of the transport device 50, and (vi) a medical chart information. (v) The location of the transport device that carries the medical device from (ii) the storage location to (i) the medical procedure location. (vi) The medical chart information includes a type of the medical procedure to be performed on the examinee.

The DB information management unit 30 includes a knowledge information memory 31, a device information memory 32, a reservation information memory 33, an examination location information memory 34, a storage space information memory 35, and a transport device information memory 36. The DB information management unit 30 includes a communication device, not shown, and is capable of obtaining various types of information from a server of an in-hospital system, or cloud computing, not shown. Moreover, the communication device is capable of receiving the examination location information from the location information communication devices 71A, 71B. Moreover, the DB information management unit 30 may obtain various types of information by a user operation on an input operation section (input interface) such as a keyboard, a touch a screen, a switch, a button, not shown. The DB information management unit 30 stores the obtained information in the memories 31 to 36, respectively, in the form of databases.

The DB information management unit 30 obtains knowledge information that is information including knowledge about an examination or a treatment for a disease, and stores the knowledge information in the knowledge information memory 31. For example, the knowledge information is a record of a flow of various examinations and treatments, such as upper endoscopy or lower endoscopy. Information about the above-mentioned preparation made by the examinee before an examination may also be important knowledge. Of course, information about food that can be eaten after an examination is also important knowledge.

The DB information management unit 30 obtains device information about a medical device that is present in the hospital, and stores the device information in the device information memory 32. The device information is organized and recorded information about devices used for each examination/treatment. Note that the device information memory 32 may also store information about a substitute medical device for a predetermined medical device. Furthermore, the device information memory 32 may hold information about a disposal location where a medical device is to be disposed of after use or a cleaning reception location where a medical device is to be cleaned after use, information about a time period for cleaning, and the like. The DB information management unit 30 allows a search regarding a device, medicine, or consumables to be used in each examination. Note that each information may be corrected based on the profile information such that the number of the device or the amount of medicine may be increased, reduced, or changed according to characteristics of each patient.

Especially, when advance preparation such as fasting or resting is necessary, such information may attract a medical worker' attention or may be notified to the patient depending on the examination. In the case of digestive endoscopy, a medicine for discharging residue in a digestive tract may be used in combination, and such discharge takes time, and notification may be made by taking the time into consideration.

The DB information management unit 30 obtains reservation information about a reservation for an examination or a treatment, and stores the reservation information in the reservation information memory 33. The DB information management unit 30 obtains the examination location information, and stores the examination location information in the examination location information memory 34. Note that in the case where there are a plurality of examination locations, the examination location information memory 34 may hold information about the availability of each examination location, for example. The DB information management unit 30 obtains the storage location information, and stores the storage location information in the storage space information memory 35. The DB information management unit 30 obtains information about the transport device 50, that is, transport device information including the transport device location information, and stores such information in the transport device information memory 36.

As described above, the DB information management unit 30 obtains information about a relevant examination location, storage space, or transport device. Note that the examination location information includes information indicating which examination location is suitable or available for which examination or treatment, information about a size of an examination location, information about equipment in an examination location, information about basic specifications of equipment, and the like.

The schedule management unit 40 may be configured with at least one processor that uses a CPU and the like. The schedule management unit 40 may operate according to a program stored in a memory, not shown to control each unit, or some or all the functions may be implemented by an electronic circuit of hardware, by installed application or by installed program. A schedule creation unit 41 in the schedule management unit 40 controls the entire medical procedure preparation apparatus 10 in an overall manner. The schedule creation unit 41 controls each unit of the medical procedure preparation apparatus 10 according to a user operation on an operation section, not shown. The schedule creation unit 41 creates schedule information for a medical procedure based on each information stored in the medical chart information management unit 20 and the DB information management unit 30.

For example, the schedule creation unit 41 creates, in relation to a schedule of an examination or a treatment to which an examinee is to be subjected at the medical procedure performing location (examination location), a schedule such as a visiting time of the examinee, transport start time of a medical device, examination start time, and the like. In the present embodiment, the schedule creation unit 41 creates a schedule by taking into account a current position of a medical worker who is to carry a medical device, and also by taking into account a time period to prepare the medical device that is to be transported from the storage space to the examination location.

The schedule management unit 40 includes a reservation unit 42, and the reservation unit 42 makes various reservations based on a created schedule. A display control unit 43 is controlled by the schedule creation unit 41, and generates display data based on information about a reservation or a schedule created by the schedule management unit 40, and provides the display data to the display unit 51 or a display device, not shown. Furthermore, the display control unit 43 may be controlled by the schedule creation unit 41 to generate display data for displaying various menu displays or input forms for preparation of examination/treatment. Accordingly, a schedule of a medical procedure and a reservation status based on the schedule may be checked on the display unit 51 or a display screen of a display device, not shown.

### (Effects)

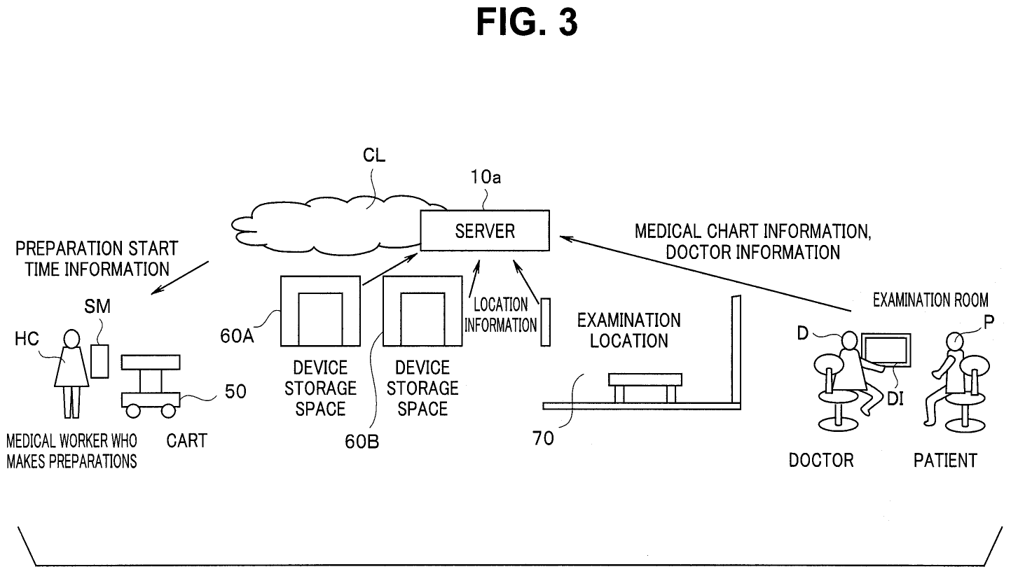

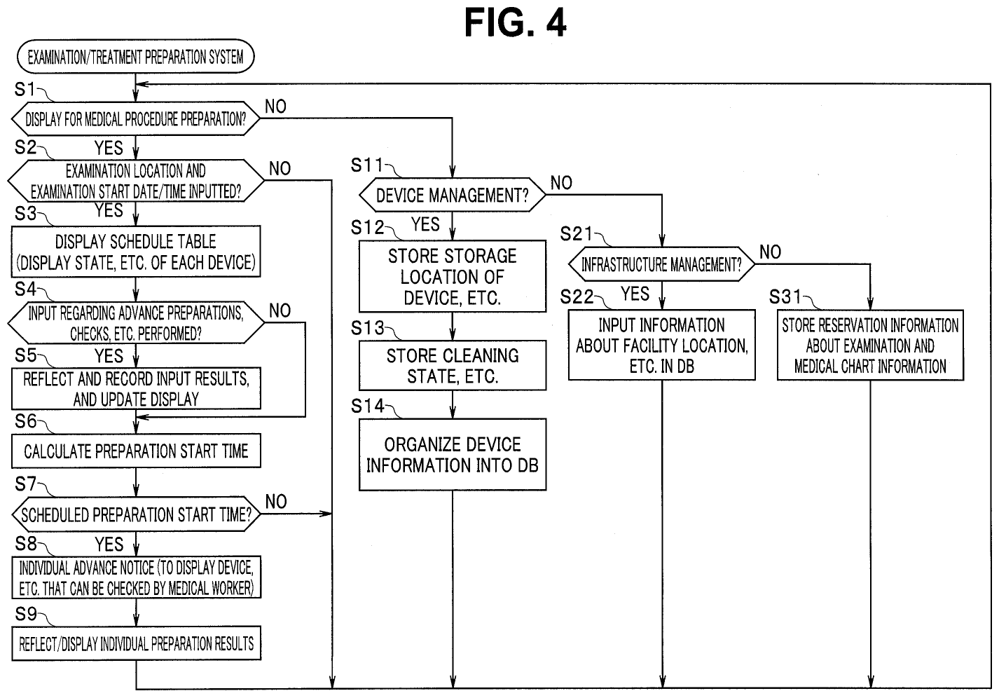

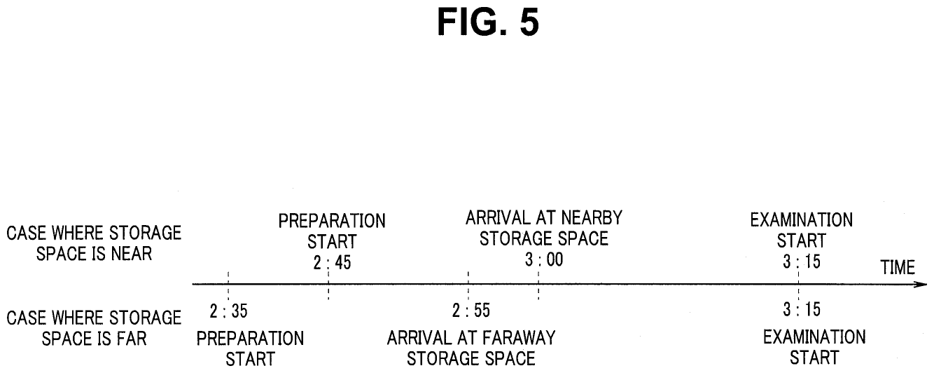

Next, an operation of the embodiment configured in the above manner will be described with reference to FIGS. 3 to 8. FIG. 3 is an explanatory diagram for describing preparation for performance of a medical procedure. FIG. 4 is a flowchart for describing an operation according to the first embodiment. Note that the flow in FIG. 4 shows a flow that is performed by the schedule creation unit 41 that controls the entire medical procedure preparation apparatus 10 in an overall manner, but each control may be performed under cooperation with distributed microcomputers or the like. Furthermore, FIG. 5 is an explanatory diagram for describing a schedule of a medical procedure.

FIG. 3 shows an example where the medical procedure preparation apparatus 10 (not shown in FIG. 3) is provided on a server 10a installed on a cloud CL. The storage location information that is information about locations of the device storage spaces 60A, 60B, the transport device location information about the transport device 50, such as a cart, and the examination location information that is information about the examination location 70 are stored in the medical procedure preparation apparatus 10 configured in the server 10a.

A doctor D examines a patient (examinee) P in a consultation room, and creates medical chart information according to matters decided by talking to the patient P, for example. The medical chart information including the examinee information and the examination/treatment information and the doctor information are provided to and stored in the medical procedure preparation apparatus 10. The medical chart information includes information about details of an examination.

To prepare for a medical procedure, the medical procedure preparation apparatus 10 determines a schedule such as a time period to prepare the medical device and a time of examination or a treatment, based on various types of information that are obtained. FIG. 4 shows a flow for preparation for a medical procedure. The flow in FIG. 4 shows an example where operation of the medical procedure preparation apparatus 10 can be controlled by user operation on an operation section, not shown, but various types of information may be obtained and a schedule may be created by controlling the medical procedure preparation apparatus 10 to provide instructions to the schedule creation unit 41 in the schedule management unit 40 of the medical procedure preparation apparatus 10 through a terminal device, not shown operated by the doctor or the like.

In S1 in FIG. 4, the schedule creation unit 41 determines whether there is an instruction to display medical procedure preparation. In addition to the display for medical procedure preparation, the schedule creation unit 41 may display information management. The display for information management is a display for obtaining information to create a schedule for a medical procedure, or in other words, each information to be stored in the medical chart information management unit 20 and the DB information management unit 30 of the medical procedure preparation apparatus 10. If there is an instruction to display the information management (NO at S41), then the schedule creation unit 41 determines whether there is an instruction for device management (S11). Furthermore, if there is no instruction for device management (NO at S11), then the schedule creation unit 41 determines whether there is an instruction for infrastructure management (S21).

If there is no instruction for infrastructure management (NO at S21), the schedule creation unit 41 stores the examination reservation information and the medical chart information in the medical chart information management unit 20 and the DB information management unit 30 (S31), and the process returns to S1. In other words, the medical chart information management unit 20 updates the examinee information memory 21 and the examination/treatment information memory 22 based on the obtained medical chart information. The DB information management unit 30 updates the reservation information memory 33 based on the examination reservation information.

If there is an instruction for infrastructure management (YES at S21), then the schedule creation unit 41 stores information about a facility location and the like in the DB information management unit 30 at S22, and the process returns to S1. A facility location is a location of the device storage space 60, the examination location 70, the station 2 (a placement location of the transport device 50, a location of the transport device 50), or the like. In other words, the DB information management unit 30 updates the examination location information memory 34, the storage space information memory 35, and the transport device information memory 36 based on such information.

If there is an instruction for device management (YES at S11), then the schedule creation unit 41 stores information about the storage location of a device and the like in the DB information management unit 30 at S12. In other words, the DB information management unit 30 stores information about the storage location, function, and the like of each medical device in the device information memory 32. Furthermore, at S13, the schedule creation unit 41 stores a cleaning state (cleaning status) and the like of the medical device in the device information memory 32. At S14, the schedule creation unit 41 organizes the device information into a database in the DB information management unit 30, and the process returns to S1. In other words, information about an examination or a treatment in which each medical device can be used and information about a storage location of each medical device are organized. Accordingly, a medical device used for a predetermined examination, as well as a storage location and a storage state of the medical device can be easily searched. Moreover, information indicating that a certain medical device is not yet stored, information about delivery date/time regarding a medical device that is already purchased but not yet delivered, and the like are also stored.

Note that the medical procedure preparation apparatus 10 also updates the doctor information and the knowledge information to latest information as appropriate, and stores each information in the doctor information memory 23 and in the knowledge information memory 31, respectively.

If there is an instruction to display medical procedure preparation (YES at S1), the schedule creation unit 41 determines, at S2, whether an examination location and an examination start date/time are inputted. Such an input may be obtained by operation of an operation section, not shown provided on the medical procedure preparation apparatus 10, or may be obtained through acquisition, by the doctor, of information that is transmitted by a terminal device, not shown installed in the consultation room, for example. When the examination location and the examination start date/time are inputted (YES at S2), the schedule creation unit 41 creates the schedule, and displays a schedule (S3). The schedule display also includes information about a state of each device, for example. The display is configured to display the display image.

The determined schedule includes information about time management indicating who is to perform which medical procedure on whom at which location, with which medical device, at what time. The created schedule information is stored in the medical procedure preparation apparatus 10. The medical procedure preparation apparatus 10 makes a reservation for the examination location, the medical device, the doctor D, a medical worker HC, and the like according to the schedule information that is stored, and transmits the schedule information to the doctor D, the medical worker HC who is to prepare the medical device, the transport device 50, and the like. For example, the schedule is displayed on a display device installed in the consultation room or the like to be checked by the doctor D, the display unit 51 arranged on the transport device 50, and the like. The medical procedure preparation apparatus 10 may cause the schedule to be displayed on a mobile terminal, such as a smartphone, of the medical worker HC, or on a display device that can be checked by the patient, the doctor, or other medical workers. The display device may be a wearable terminal or the like.

For example, the schedule information that is transmitted to the medical worker HC who prepares the medical device includes preparation start time information. The schedule information also includes information indicating the storage location to which the medical worker HC is to move the transport device 50, such as a cart, and the medical device that is to be placed on the transport device 50 to be carried. According to the information displayed on the display unit 51 or the like, the medical worker HC can smoothly perform a preparation task regarding the medical device.

The schedule creation unit 41 of the medical procedure preparation apparatus 10 controls the DB information management unit 30 and searches through the knowledge information memory 31 based on the medical chart information created by the doctor D, and thereby obtains information about a medical device used for the medical procedure, and performs a search, in the device information memory 32, regarding the medical device present in the hospital. The schedule creation unit 41 accesses the examination location information memory 34, the storage space information memory 35, and the transport device information memory 36, and calculates a time period to move a medical device to be used for the medical procedure to the medical procedure performing location.

For example, the schedule creation unit 41 calculates distances among the transport device 50, the device storage space 60, and the examination location 70 based on information about the position where the transport device 50 is located (the transport device location information), the storage location information about the device storage space 60, and the medical procedure performing location information about the examination location 70, and calculates the time period to transport the medical device based on an average movement speed (movement speed) of the transport device 50, or in other words, an average movement speed at which the medical worker HC moves the transport device 50.

For example, an examination start time point that is a scheduled performing time (performing time) of a medical procedure is set to 15:15. FIG. 5 describes a schedule that is created by the medical procedure preparation apparatus 10 in such a case. For example, in the case where the placement location of the transport device 50 is the station 2 in FIG. 2, in order to carry a medical device to the examination location 70, the medical worker HC first needs to move the transport device 50 from the station 2 to the device storage space 60, place the medical device on the transport device 50 in the device storage space 60, and move the transport device 50 where the medical device is placed to the examination location 70.

A medical procedure preparation system comprises the input interface, the display 51, and at least one processor comprising hardware 40. The at least one processor 40 is configured to receive an input containing (i) the medical procedure location where the medical procedure is performed, and (ii) the storage location where a medical device is stored, calculate (iii) the distance between (i) the medical procedure location and (ii) the storage location, calculate one of (a) the preparation start time of the medical device used in the medical procedure and (b) the performing time of the medical procedure using the medical device, and output a display image indicating one of (a) and (b) to the display.

Calculation one of (a) and (b) can be based on (iii) the distance and (iv) the movement speed of the transport device 50. Calculation one of (a) and (b) can be further based on (vi) a location of the transport device 50 that moves between (i) the medical procedure location and (ii) the storage location. Calculation one of (a) and (b) can be based on (v) the cleaning status of the medical device.

When the at least one processor can be configured to calculate (a), calculation of (a) can be further based on (b), or when the at least one processor can be configured to calculate (b), calculation of (b) can be further based on (a). The display image can indicate (i) the medical procedure location and (ii) the storage location.

An upper section in FIG. 5 shows an example where the device storage space 60 is relatively close to the station 2 and the examination location 70, and a lower section shows an example where the device storage space 60 is relatively far from the station 2 and the examination location 70. As shown in the upper section in FIG. 5, for example, in the case where the device storage space 60 is relatively close to the station 2 and the examination location 70, the medical worker HC starts preparation, or in other words, starts to move the transport device 50, at 14:45, and arrives at the device storage space 60 that is relatively close at 15:00. The medical worker HC places the medical device on the transport device 50 in the device storage space 60, and then, carries the medical device to the examination location 70. An examination can thus be started at the examination location 70 at 15:15.

In the same manner, as shown in the lower section in FIG. 5, for example, in the case where the device storage space 60 is relatively far from the station 2 and the examination location 70, the medical worker HC starts to move the transport device 50 at 14:35, and arrives at the device storage space 60 that is relatively far at 14:55. The medical worker HC places the medical device on the transport device 50 in the device storage space 60, and then carries the medical device to the examination location 70. An examination can thus be started at the examination location 70 at 15:15.

As described above, the schedule creation unit 41 may determine a relationship between a scheduled performing time of a medical procedure and a scheduled preparation start time of a medical device based on a distance between the medical procedure performing location and the storage location and a movement speed of the transport device 50. By using the relationship, the schedule creation unit 41 may determine the scheduled preparation start time of a medical device corresponding to the scheduled performing time of a medical procedure, or may determine the scheduled performing time of a medical procedure corresponding to the scheduled preparation start time of a medical device.

Furthermore, some medical devices to be moved from a storage location need cleaning, and a cleaned medical device must be used. For example, a scope section of an endoscope apparatus is inserted inside a body of an examinee to perform examination, and thus, cleaning must be completed before the endoscope apparatus is transported to the medical procedure performing location (an endoscopy location). It is important that cleaning be completed before transport.

Moreover, the schedule creation unit 41 may store, for each medical procedure performing location, an average time period to carry a medical device from the placement location of the transport device 50 via each device storage space 60 in the form of a database, and perform scheduling based on the location database. For example, the location database may be stored in the DB information management unit 30.

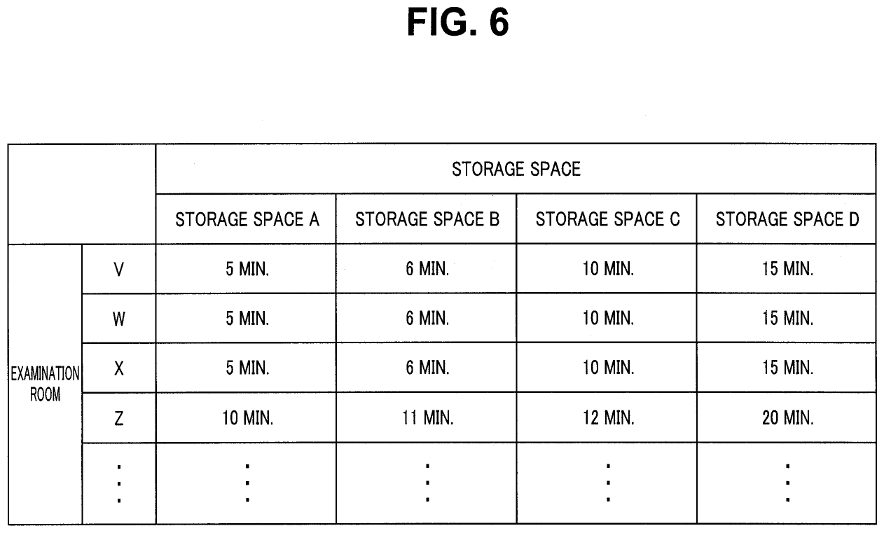

FIG. 6 is an explanatory diagram showing an example of the location database.

The example in FIG. 6 shows a time period for preparation of a medical device in relation to examination rooms V, W, X, Z and storage spaces A to D. For example, it can be seen that an average time period by the medical worker HC to move the transport device 50 from the placement location of the transport device 50 to the storage space B, place a specified medical device on the transport device 50 in the storage space B, and carry the medical device to the examination room Z is 11 minutes.

Here, as with the “storage location” described above, the placement location of the transport device 50 may imply a location where the transport device is contained for a specific period of time, but in the present disclosure, the placement location also refers to a location where the transport device contained or stored is taken out and kept until a medical procedure is actually performed. This is because, although in many facilities, the placement location serves also as a preparation (standby) location, in some facilities, the transport device is taken out of the placement location in advance and is placed in a transport device standby location, and such a case may also be considered short-term storage.

As described above, the schedule creation unit 41 is capable of performing scheduling using a database of transport time period to each examination room and each storage space. Note that the example in FIG. 6 shows an example of a database related to time, but scheduling may also be performed using a database related to each distance.

Furthermore, the schedule creation unit 41 may add a check function or the like to be used by a user to the schedule display, and perform schedule display that more effectively supports the preparation task for the medical procedure.

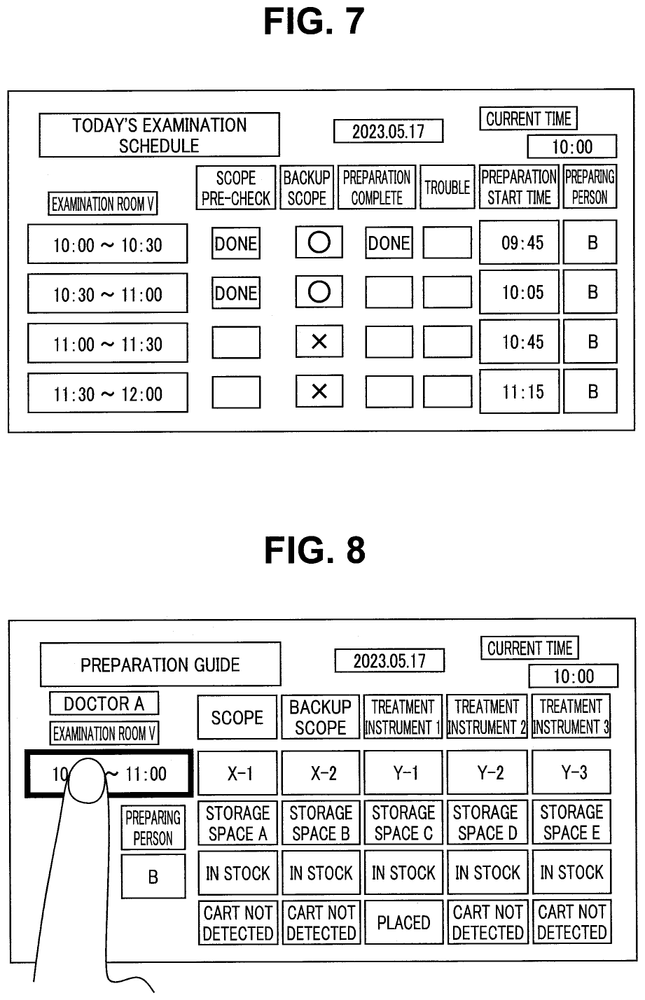

FIG. 7 is an explanatory diagram showing an example display of schedule display including the check function. FIG. 7 shows the schedule display for a specific medical procedure performing location. Furthermore, FIG. 8 shows a preparation guide display for a specific medical procedure (examination). The displays in FIGS. 7 and 8 may be displayed on a mobile terminal carried by the medical worker HC, for example. When the examination location and the examination start date/time are inputted at S2 in FIG. 4, the schedule display in FIG. 7 is displayed. Pre-check and the like may be performed using the schedule display in FIG. 7, and a result of checking whether there is failure of a device, for example, may be inputted on the morning of the day of the examination to ensure that troubles do not arise immediately before the examination.

The example in FIG. 7 is a schedule for the examination room V, and the state of a medical procedure in the examination room V on the day may be grasped from the display in FIG. 7. The display in FIG. 7 includes a date display H1 and a current time display H2. In FIG. 7, one row shows the state of one examination, and it is indicated that examinations are performed on May 17, 2023, from 10:00 to 10:30, from 10:30 to 11:00, from 11:00 to 11:30, and from 11:30 to 12:00. Note that the current time is 10:00.

At S4, the schedule creation unit 41 determines whether input is performed regarding advance preparation, check, and the like. In FIG. 7, “scope pre-check” indicates whether a scope is checked in advance. Preparation for an examination (dietary restriction, scheduling, etc.) is burdensome for an examinee, and thus, “scope pre-check” of checking an endoscope, which is a medical device, is performed in advance to ensure that the examination can be performed without fail. Note that “scope pre-check” is performed, for example, in the device storage space 60, before the examination, such as on the morning of the examination day.

If the schedule creation unit 41 determined that input is performed regarding advance preparation, check, and the like (YES at S4), then the schedule creation unit 41 reflects the input result and updates the record and the display (S5). In the example in FIG. 7, the state of both the scope pre-check from 10:00 to 10:30 and “scope pre-check” from 10:30 to 11:00 is changed to “done”, and it can be seen that the scope pre-check is completed.

Furthermore, a backup endoscope may be prepared in case an accident, such as when an endoscope is broken during a medical procedure and becomes unusable. “Backup scope” indicates whether such a backup endoscope is prepared, and in FIG. 7, a circle indicates that a backup endoscope is prepared. “Preparation complete” indicates whether preparation of the medical device is complete and the medical procedure can be performed, and in FIG. 7, “done” indicates that the preparation is complete. “Trouble” indicates whether there is a trouble, and details of the trouble. Note that if no input operation is detected at S4, the process proceeds to S6.

The schedule creation unit 41 calculates the preparation start time at S6, and updates the display. “Preparation start time” in FIG. 7 indicates a time when the medical worker HC starts preparation of the medical device, or in other words, a time when the medical worker HC is to start moving the transport device 50 to carry the medical device. The example in FIG. 7 indicates that the preparation may be started at 9:45 for the examination that is to be performed at 10:00. “Preparing person” shows the name of the medical worker HC who is to prepare the medical device. Moreover, before endoscopy, the examinee needs to be prepared by cleaning an examination part. Preparation often takes time. For example, the examinee starts fasting from previous day or starts taking a medicine for a pretreatment, such as intestinal cleansing, half a day in advance. If the preparation is not correctly performed, then the examination or the treatment will not be correctly performed. Accordingly, it is important to create a schedule, and to inform the examinee and the medical worker of the schedule. Such an event may be said to be calculation or determination of the preparation start time in a broad sense. Such preparation start time may be calculated in relation to a scheduled time of an examination or a treatment, based on preparation time information for an examinee recorded in association with the examination or the treatment.

Note that the preparation start time may be already calculated at a stage of display of the schedule display at S3, and calculation of the preparation start time at S6 may be the second calculation. With the system, the examination room and the examination time are associated with the corresponding examination, the patient, the primary doctor, and the preparing person, and a device (medical device) used for the examination or the treatment is checked in the database. The time when the preparation is to be started may be determined at S3 based on a distance to the storage location of the device. Accordingly, the preparation start time may be displayed from the start of schedule display.

However, if the result of a pre-check indicates that there is a device that cannot be used, a schedule may become different from an initial schedule since a person needs to go to another storage space or go to a location where an ordered medical device is delivered, and, as a result, the preparation start time is changed. The preparation start time may be changed due to the medical device being cleaned earlier than the scheduled time or due to cleaning not being completed. Calculation of the preparation start time at S6 assumes such a possibility of change. The at least one processor can be configured to calculate one of (c) an updated preparation start time of the medical device and (d) an updated performing time of the medical procedure. Calculation one of (c) and (d) can be based on (iii) the distance, (iv) the movement speed of the transport device, (v) a preparation status that is already prepared and placed on the transport device 50 and (vi) the location of the transport device 50 that moves between (i) the medical procedure location and (ii) the storage location. The at least one processor can be configured to output the first display image indicating one of (a) and (b) to the display, output a second display image indicating (c) the updated preparation start time to the display, and switch the first display image and the second display image.

In the case of a medical device, such as an endoscope, where cleaning is important, the present application is quite effective. Thanks to the present application, “endoscopy preparation apparatus” can be provided, and a (scheduled) cleaning start time for an endoscope apparatus that is to be used in endoscopy or a scheduled preparation start time for transport of the endoscope apparatus may be determined based on endoscopy performing location information that is information about a location where endoscopy is performed and storage location information that is information about the storage location of the endoscope apparatus. Such a process may be performed by at least one processor through execution of a specific program or the like. Of course, both the scheduled cleaning start time and a transport preparation start schedule may be determined. In such a case, if the scheduled transport time is too early and cleaning is not finished, time is wasted. Thus, a schedule that takes coordination into account is desirable. When cleaning is delayed past the scheduled cleaning start time, the examination will be delayed, and thus, an instruction may be changed to indicate use of another device.

Note that if it becomes clear at S5 that there is a trouble with the medical device, the schedule creation unit 41 may issue a request to perform repair or to place a new order, for example. Also in such a case, there may be a case where the repair is not completed or delivery is not performed within the scheduled time, but results of information input at S12, S13 are reflected according to the situation, and the schedule can be prevented from being changed as much as possible.

At S7, the schedule creation unit 41 determines whether the scheduled preparation start time is reached. When the scheduled preparation start time is reached (YES at S7), the schedule creation unit 41 provides individual advance notice (alarm) indicating that a task is to be started, to a display device or the like, that can be checked by the medical worker HC (S8). The medical worker HC may thus reliably grasp that the preparation of the medical device should be started. Note that if NO is determined at S7 or S2, the process returns to S1. In these steps, display or announcement is performed in relation to preparation of apparatus, devices and the like, for example, but an improvement may be made to display an advice for the examinee or to transmit the advice to a mobile terminal of the examinee. Of course, a guide may be provided to the doctor or the medical worker in relation to preparation for the examination or the treatment, a movement route, or the examination room.

At S9, the schedule creation unit 41 causes a preparation result indicating whether each medical device is prepared to be reflected in the display in FIG. 8. FIG. 8 shows specific details of each examination. For example, when the medical worker HC performs an operation of specifying a display position indicating a time slot of an examination in FIG. 7, a preparation guide display in FIG. 8 that is a list of medical devices to be prepared is displayed. FIG. 8 is an example where a second examination that is to be performed from 10:30 to 11:00 is specified by the medical worker HC. In other embodiment, the operation of specifying a display position indicating can be including specifying the today's examination schedule, the type of the medical procedure to be performed on the examinee, the medical procedure location, the storage location, the performing time of the medical procedure, the medical chart information, the preparation status that is already prepared and placed on the transport device, the cleaning status of the medical device, the trouble information indicating whether there is a trouble with the medical device or the backup scope indicating whether such a backup endoscope is prepared, or the preparing person information indicating the name of the medical worker HC who is to prepare the medical device.

The example in FIG. 8 is a guide for performing preparation for an examination that is to be performed in the examination room V from 10:30 to 11:00 by a doctor A. Thanks to the display in FIG. 8, the medical worker HC may grasp various medical devices to be prepared. In other words, in the example in FIG. 8, the type of medical device to be used in the examination, the storage location, whether there is stock, and whether the medical device is placed on the transport device 50 may be grasped. In other words, the example in FIG. 8 indicates that the type of scope indicated by “scope” is X−1, the scope is stored in “storage A”, there is stock of the scope, and the scope is not yet placed on the transport device 50. In the same manner, the type, the storage location, whether there is stock, whether placed on the transport device 50 are indicated with respect to a backup scope indicated by “backup scope” and treatment instruments indicated by “treatment instrument 1” to “treatment instrument 3”. Note that FIG. 8 also shows that the preparing person is “B”.

The medical worker HC whose name is “B” can grasp the medical device that he/she has to prepare, by checking the display in FIG. 8. Furthermore, the medical worker HC can grasp the storage location of each medical device to be prepared, and whether there is stock of the medical device. Furthermore, the transport device 50 is provided with the placement check unit 52, and when the medical worker HC places a medical device on the transport device 50 at the storage location, the placement check unit 52 may detect placement of the medical device, and change the display in FIG. 8 from “cart not detected” to “placed” or change the color. The example in FIG. 8 indicates that a Y−1 type treatment instrument indicated by “treatment instrument 1”, is already placed on the transport device 50 in “storage space C”.

Note that when the placement check unit 52 and the schedule creation unit 41 operate in conjunction with each other, and the transport device 50 moves away from the storage location without the medical device being placed, control may be performed such that an alarm is issued from a speaker, not shown. Furthermore, the stock DB apparatus 63A, 63B or the like in the device storage space 60 may be provided with the same function as the function of the placement check unit 52. In such a case, the device storage space 60 may operate in conjunction with the schedule creation unit 41 to present information about the medical device to be placed on the transport device 50, to the medical worker HC.

As described above, whatever is prepared and placed on the transport device 50 is reflected on the display, and a preparing person may efficiently perform preparation without fail, and the examination/treatment may be performed on time with no redo or the like. When the examination room is small, and the schedule is tight, such timely preparation increases efficiency of the medical procedure. Reducing stress on doctors and medical workers by saving troubles is a major topic in the medical field. Moreover, although not described herein, with the system as described in the present application where the location of a device is managed, even when a different device is used during an examination, the location of the device may be searched for, and arrangement may swiftly be made.

Thanks to the displays shown in FIGS. 7 and 8, the medical worker HC is able to easily determine the schedule of an examination and the medical device used for the examination. Furthermore, the time point when transport is to be started is also displayed, and the schedule may be easily managed. Moreover, the storage location of a medical device used for a medical procedure, whether there is stock, and whether the medical device is placed on the transport device 50 may be easily checked, and preparation of the medical device may be effectively supported, and the medical device may be reliably prepared.

In other words, the displays in FIGS. 7 and 8 give the medical worker HC a clear instruction as to a task that is to be performed on the day, and the medical worker HC may reliably perform the medical procedure without fail by checking the displays. Furthermore, although not shown, before endoscopy, an examinee needs to be prepared by cleaning an examination part. Preparation often takes time. For example, the examinee starts fasting from previous day or starts taking a medicine for a pretreatment, such as intestinal cleansing, half a day in advance. If the preparation is not correctly performed, then the examination or the treatment will not be correctly performed. Accordingly, it is important to create a schedule, and to inform the examinee and the medical worker of the schedule. Such an event may be said to be calculation or determination of the preparation start time in a broad sense. It can be said that the medical procedure preparation apparatus of the present example emphasizes to include at least one processor configured to determine a scheduled performing time of a medical procedure and a scheduled preparation start time (including an intestinal cleansing start time) of the medical procedure, based on the medical procedure performing location information that is information about the location where the medical procedure is performed and arrival/departure location information that is information about arrival/departure location of a medical device to be used in the medical procedure. However, other examples also include the same disclosure. Calculation one of (a) and (b) can be further based on arrival/departure location information indicating an arrival/departure location of the medical device between (i) the medical procedure location and (ii) the storage location.

As described above, in the present embodiment, preparation for a medical procedure is effectively supported by creating a schedule for performing the medical procedure, based on a positional relationship between the service provision location of the medical procedure and the storage space of the medical device. Accordingly, medical workers including the doctor are able to smoothly perform preparation at the time of a medical procedure, and an increase in burden on the examinee may be prevented. Moreover, description of a movement time of the doctor to move to the examination room is omitted, but the schedule may be created taking such a movement time into account. In other words, in such a case, the medical procedure preparation apparatus includes at least one processor, and may determine a candidate for the scheduled performing time of the medical procedure based on the medical procedure performing location information that is information about the location where the medical procedure is performed, the storage location information that is information about the storage location of a medical device to be used in the medical procedure, and a schedule of the doctor who performs the medical procedure.

The at least one processor can be configured to output the first display image indicating one of (a) and (b) to the display, output the second display image indicating the medical device that is already prepared and placed on the transport device, and switch the first display image and the second display image.

### Modification

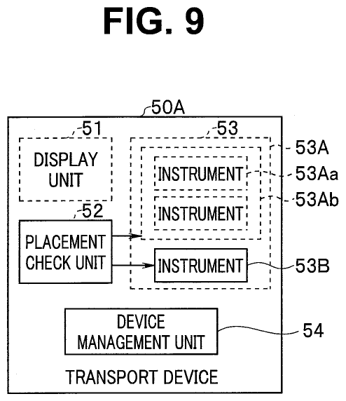

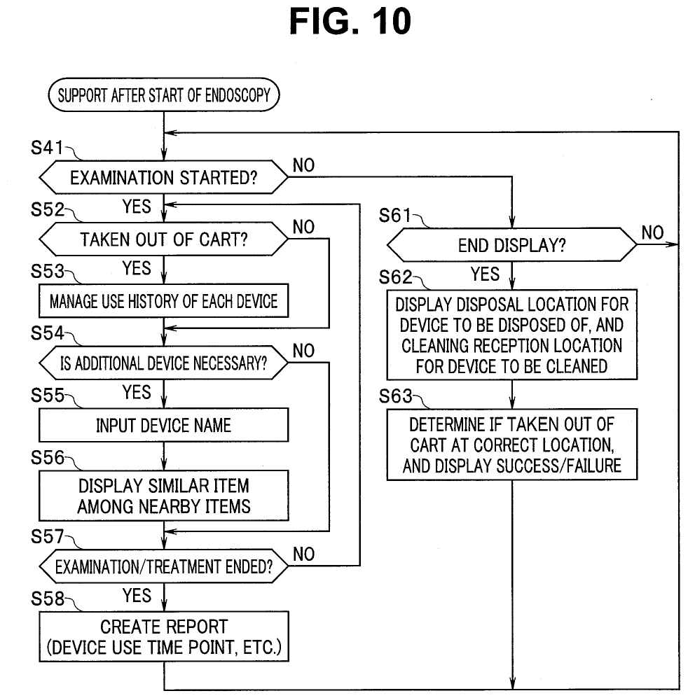

FIGS. 9 and 10 are related to a modification, and FIG. 9 is a block diagram showing a transport device 50A that is adopted in the modification. In FIG. 9, components that are same as the components in FIG. 1 are denoted by the same reference signs, and description will be omitted.

The medical system in FIG. 1 supports medical workers including doctors mainly in terms of preparation for examinations and treatments. The transport device 50A in FIG. 9 includes a function of giving support after an examination or a treatment. The transport device 50A in FIG. 9 is different from the transport device 50 in FIG. 1 in that the transport device 50A includes a device management unit 54. The device management unit 54 may be configured with at least one processor that uses a CPU and the like. The device management unit 54 may operate according to a program stored in a memory, not shown to control each unit, or some or all the functions may be implemented by an electronic circuit of hardware, by installed application or by installed program. Needless to say, the at least one processor, the memory and the like may function on a cloud or may function in a distributed manner, or various other applications are possible.

The device management unit 54 can manage a state of use of each medical device, for example. Furthermore, by operating in conjunction with the schedule creation unit 41, the device management unit 54 implements a function of displaying, on various display devices such as the display unit 51, a location where each medical device is disposed of after use, a location where each medical device is cleaned, and the like.

FIG. 10 is a flowchart for describing an operation after start of endoscopy.

When the device management unit 54 determines the start of an examination at S41 (YES at S41), the device management unit 54 determines whether a medical device is removed from the transport device 50, such as a cart (S52). Such determination may be performed based on information from a camera configured to capture the instrument 53 that is placed on the transport device 50, information from a communication device configured to read information in an IC tag or the like provided on the instrument 53, or an input operation by the medical worker HC or the like. When the medical device is removed from the transport device 50 (YES at S52), the device management unit 54 determines that the device is used, and updates and manages a use history of the device (S53). Note that the device management unit 54 proceeds to S54 without updating the use history of a medical device that is not removed from the transport device 50.

At S54, the device management unit 54 determines whether an additional medical device is necessary. For example, the device management unit 54 determines whether an additional medical device is required, based on an input operation or the like from the medical worker HC according to an instruction from the doctor. If an additional medical device is required (YES at S54), the device management unit 54 receives an input operation from the medical worker HC regarding a device name (S55), and displays a substitute (similar item) for the specified medical device among medical devices placed on the transport device 50 (S56). When the device management unit 54 determines that an additional medical device is not required, the process proceeds to S57.

At S57, the device management unit 54 determines end of the examination or the treatment. When there is no instruction to end the examination or the treatment, the device management unit 54 returns to S52, and when there is an instruction to end, the device management unit 54 creates a report regarding the use of each medical device (S58), and the process is returned to S41. The report includes information about a device use time point.

At the end of the examination, the device management unit 54 proceeds from S41 to S61, and determines whether there is an instruction for end display that is displayed at the end of the examination. When there is an instruction for end display (YES at S61), the device management unit 54 performs, at S62, display indicating a disposal location in relation to a device that is to be disposed of, and display indicating a cleaning reception location (cleaning arrival location) in relation to a device that is to be cleaned. The medical worker HC checks the display and moves the transport device 50, and may thus carry the medical device after use to an appropriate disposal location or cleaning reception location. At S63, the device management unit 54 determines the medical device that is removed from the transport device 50, determines whether the medical device is removed from the transport device 50 at a correct location, by operating in conjunction with a specified schedule creation unit 41, and displays a result of success/failure. Calculation one of (a) and (b) can be based on a time of arrival of the medical device at (i) the medical procedure location.

As described above, in the present modification, the medical device may be easily managed by determining the state of the medical device after start of an examination/treatment.

The medical procedure preparation method comprises receiving the input containing (i) a medical procedure location where a medical procedure is performed, and (ii) a storage location where a medical device is stored from an input interface, calculating (iii) a distance between (i) the medical procedure location and (ii) the storage location where the medical device is stored, calculating one of (a) a preparation start time of the medical device used in the medical procedure and (b) a performing time of the medical procedure using the medical device, and outputting a display image indicating one of (a) and (b) to a display. Calculation one of (a) and (b) can be based on (iii) the distance and (iv) a movement speed of a transport device. The medical procedure preparation method comprises receiving the input signal from the input interface, obtaining, based on the input signal, (i) the medical procedure location where the medical procedure is performed, (ii) the storage location where a medical device is stored, and (iii) the performing time of the medical procedure using the medical device, calculating a preparation start time of the medical device, and outputting the preparation start time. The medical device is used in the medical procedure. The input signal indicating a medical procedure information that can be displayed on the display in FIG. 7. The medical procedure information can be the today's examination schedule, the type of the medical procedure to be performed on the examinee, the medical procedure location, the storage location, the performing time of the medical procedure, the medical chart information, the preparation status that is already prepared and placed on the transport device, the cleaning status of the medical device, the trouble information indicating whether there is a trouble with the medical device or the backup scope indicating whether such a backup endoscope is prepared, or the preparing person information indicating the name of the medical worker HC who is to prepare the medical device. The input signal can be received by selecting the icon on the screen by the medical worker or doctor. The outputting the preparation start time can include outputting the display image indicating the preparation start time.

### Second Embodiment

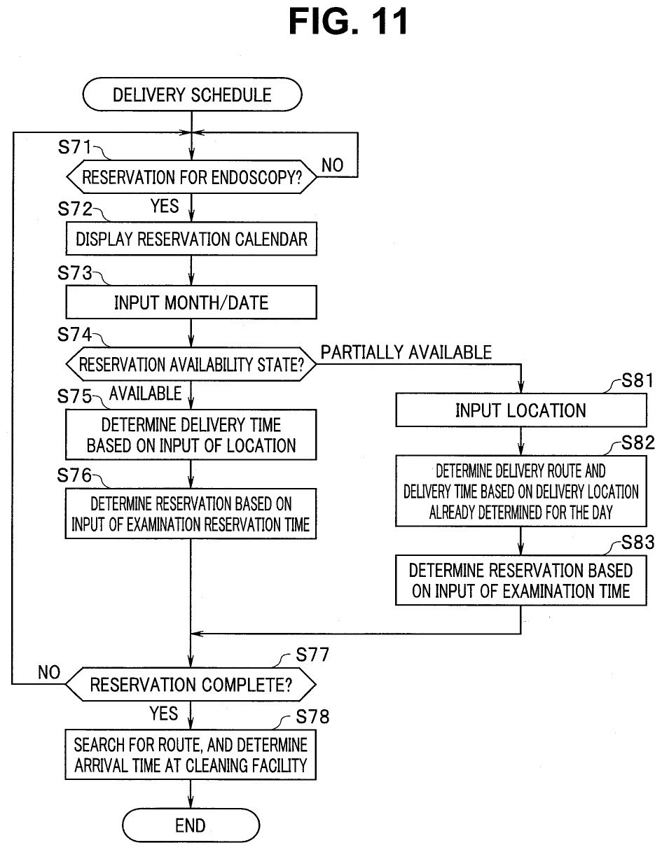

FIG. 11 is a flowchart showing an operation flow adopted in a second embodiment. A hardware configuration of the present embodiment is the same as the hardware configuration in FIG. 1. The first embodiment describes an example where the medical device is carried inside a hospital. The second embodiment is an example where the medical device is carried from a delivery center by a car. Furthermore, the present embodiment is an example where a reservation for the medical procedure is performed by the examinee.

Nowadays, thanks to reduction in the size of medical devices, medical devices that were allowed to be installed only in large hospitals are coming to be used in small- to medium-sized clinics and the like, and examinations that were performed in large hospitals can now be performed in small- to medium-sized clinics. Moreover, depending on the type of the examination, the medical device may be brought to a patient's home or the like and the examination is performed. When considering efficient use of a medical device, because cleaning or sterilization of a medical device takes time, a cleaning or sterilization process can be performed at night when the medical device is not usually needed by the patient or the medical worker. Accordingly, the schedule creation unit 41 of the present embodiment can create an optimal delivery schedule that takes into account a transport route of a medical device that starts from a delivery center of the medical device, stops one or more medical procedure performing locations where a medical procedure such as endoscopy is performed, and reaches a sterilization facility where the cleaning or sterilization process is performed.

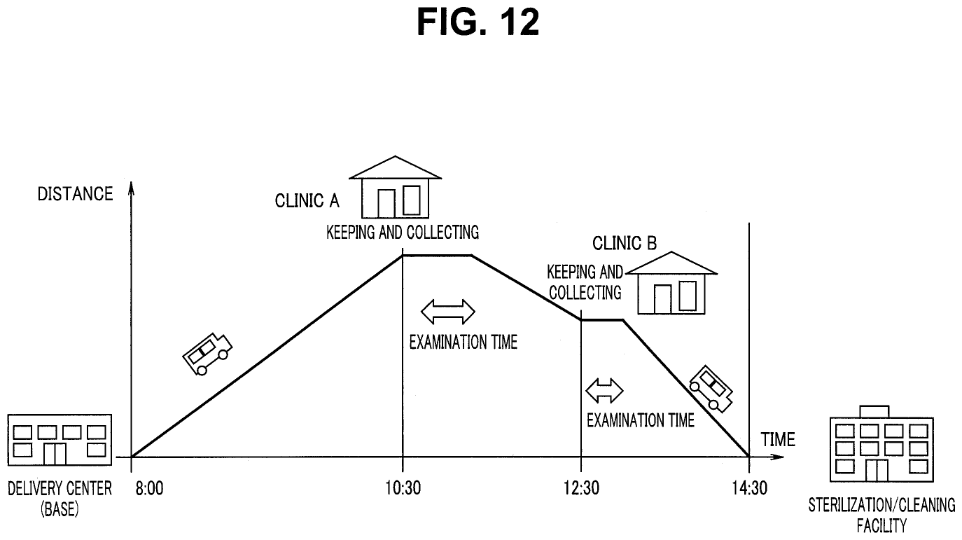

FIG. 12 is an explanatory diagram showing an example of the transport route of the medical device. FIG. 12 shows an example of a delivery schedule according to the transport route, where the horizontal axis shows time and the vertical axis shows a distance from the delivery center. Note that the example in FIG. 12 shows an example where the delivery center (corresponding to the storage space 3 in FIG. 2) and the sterilization facility are on the same premises, but the delivery center and the sterilization facility may instead be at different locations.

In the example in FIG. 12, a vehicle carrying the medical device leaves the delivery center at 8:00, and arrives at clinic A at 10:30. An examination is performed in clinic A using the medical device delivered, and the medical device is collected at the vehicle when the examination is ended. Moreover, the vehicle heads to clinic B, and arrives at clinic B at 12:30. An examination is performed in clinic B using a medical device that is delivered and that is not yet used, and the medical device is collected at the vehicle when the examination is ended. After collecting the medical devices used at clinic A and clinic B, the vehicle heads to a sterilization/cleaning facility, and arrives at the facility at 14:30. The schedule creation unit 41 creates a delivery schedule that takes into account such a delivery route.

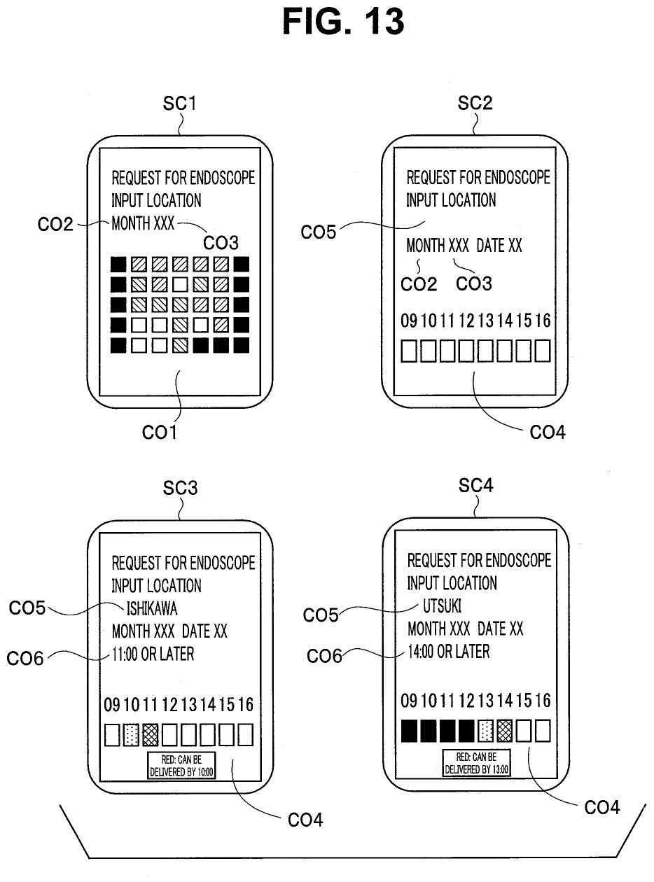

Next, an operation of the embodiment configured in the above manner will be described with reference to FIGS. 11 and 13. FIG. 13 shows an example display on a user terminal that accesses the schedule creation unit 41, and describes input operation for creation of the delivery schedule, and schedule display of the delivery schedule. Note that a smartphone or the like may be used as the user terminal. A request for delivery of a medical device may be issued by using the user terminal, by accessing the schedule creation unit 41 that is constructed as a Web application on a server on a cloud, for example. Note that a doctor may operate the user terminal to make a reservation, or an examinee himself/herself may perform a reservation operation using the user terminal.

At S71 in FIG. 11, the schedule creation unit 41 is in a state of wait for an instruction for a reservation for a medical procedure such as endoscopy. When a user activates a reservation application for endoscopy using the user terminal, the user terminal accesses the schedule creation unit 41. The schedule creation unit 41 then determines YES at S71, and causes a reservation screen SC1 with a calendar display shown in FIG. 13 to be displayed on the user terminal (S72). Note that the schedule creation unit 41 creates the reservation screen SC1 based on information stored in the medical chart information management unit 20 and the DB information management unit 30.

The reservation screen SC1 in FIG. 13 includes a reservation month list CO1 including 7×5 squares. Seven squares that are arranged horizontally indicate a reservation state for one week, from Sunday to Saturday, and five squares that are arranged vertically indicate a reservation state for each week, from first to fifth weeks. Black squares indicate days when delivery is not available, such as holidays. Squares hatched with right diagonal lines indicate days that are fully reserved and no more reservations can be made. Squares hatched with left diagonal lines indicate days when reservations can be made for some time slots, and white squares indicate days when there are no reservations. Note that the reservation state may be shown by changing display modes, such as by changing a color instead of a pattern of the square.

A user may change the month of the reservation month list CO1 by inputting a month in a month specification section CO2 on the reservation screen SC1. Moreover, the user refers to the reservation month list CO1, and inputs a desired examination day among days when a reservation can be made, to a date specification section CO3. When the schedule creation unit 41 obtains month and date at S73, the schedule creation unit 41 causes a reservation screen SC2 shown in FIG. 13 to be displayed on the user terminal, based on a reservation state stored in the reservation unit 42. Note that a reservation time display section CO4 for the reservation day is displayed at a bottom of the reservation screen SC2. Note that the reservation time display section CO4 on the reservation screen SC2 in FIG. 13 is blank, and it can be seen that there are no reservations for the day.

Furthermore, the user inputs a location in a location specification section CO5, or in other words, inputs information about an examination location (medical procedure performing location). FIG. 13 shows an example where “Ishikawa” is specified. The schedule creation unit 41 determines whether the reservation state on the specified month/date indicates available or partially available (S74). Here, the schedule creation unit 41 determines that no reservation is yet made for the specified month/date, and proceeds to S75 to determine a delivery time. In other words, the schedule creation unit 41 determines a delivery route based on a positional relationship between the position of the inputted location and the delivery center, and determines the delivery time of the medical device based on an average speed of the vehicle for carrying the medical device, a distance of the delivery route, and a traffic state.

The schedule creation unit 41 causes a reservation screen SC3 in FIG. 13 to be displayed on the user terminal, the reservation screen SC3 including information about the delivery time and a time when the examination can be performed according to the delivery time. Note that on the reservation screen SC3, it can be seen based on a dotted square (displayed in red, for example) in the reservation time display section CO4 that the medical device is scheduled to be delivered at 10:00. Furthermore, on the reservation screen SC3, it can be seen that the examination can be performed from 11:00, based on a display of an examination possible time display section CO6.

The user inputs a desired time when the examination is to be started, in the reservation time display section CO4 displayed at a bottom of the reservation screen SC3. In the example of the reservation screen SC3 in FIG. 13, a time after 11:00 is a time when an examination can be performed, and that the user wishes for the examination at 11:00 is indicated by a mesh square. The schedule creation unit 41 determines a reservation for an examination at the specified time based on input of the desired time for start of the examination, and causes the reservation unit 42 to store a reservation time (S76).

Next, the user refers to the reservation screen SC1, and makes a reservation for a day when a reservation can be made in some time slots, for example. For example, a reservation is to be made for the same month/date as for the reservation day shown on the reservation screen SC3. In such a case, the schedule creation unit 41 proceeds from S74 to S81. Moreover, when the user inputs information about the examination location in the location specification section CO5 (in FIG. 13, “Utsuki” is inputted), the schedule creation unit 41 determines the delivery route based on the location specified at S81 and a delivery location already determined for the day, and determines the delivery time of the medical device based on an average speed of the vehicle for carrying the medical device, a distance of the delivery route, and a traffic state.

The schedule creation unit 41 causes a reservation screen SC4 in FIG. 13 to be displayed on the user terminal, the reservation screen SC4 including information about the delivery time and a time when the examination can be performed according to the delivery time. On the reservation screen SC4, it can be seen, based on black squares in the reservation time display section CO4, that delivery to another delivery destination is performed from 9:00 to 12:00. Moreover, it can be seen, based on a dotted square in the reservation time display section CO4 on the reservation screen SC4, that the medical device is scheduled to be delivered at 13:00. Moreover, on the reservation screen SC4, it can be seen, based on the display in the examination possible time display section CO6, that the examination can be performed from 14:00.

The user inputs a desired time when the examination is to be started, in the reservation time display section CO4 displayed at a bottom of the reservation screen SC4. In the example of the reservation screen SC4 in FIG. 13, that the user wishes for the examination from 14:00 is indicated by a mesh square. The schedule creation unit 41 determines a reservation for the examination at the specified time, based on input of the desired time for start of the examination, and causes the reservation unit 42 to store the reservation time (S76). Furthermore, before endoscopy, an examinee needs to be prepared by cleaning an examination part. Preparation often takes time. For example, the examinee starts fasting from previous day or starts taking a medicine for a pretreatment, such as intestinal cleansing, half a day in advance. If the preparation has to be correctly performed, then the examination or the treatment will not be correctly performed. Accordingly, it is important to create a schedule and to inform the examinee and the medical worker of the schedule. Such an event may be said to be calculation or determination of the preparation start time in a broad sense. Such preparation time information is categorized and recorded in a database for each examination or for each treatment that is assumed.

Note that the present example indicates an example where a doctor in a clinic performs endoscopy, but a specialist may move together with the endoscope or by other transport means.

At S77, the schedule creation unit 41 determines whether reservation is completed. If reservation is not completed, the schedule creation unit 41 returns to S71, and if reservation is completed, the schedule creation unit 41 proceeds to S78. At S78, the schedule creation unit 41 searches for a route to the sterilization/cleaning facility, determines an arrival time at the sterilization/cleaning facility, and causes the reservation unit 42 to store the arrival time. The schedule creation unit 41 notifies the sterilization/cleaning facility of information about the arrival time at the sterilization/cleaning facility.

As described above, in the present embodiment, the delivery route is determined based on a positional relationship among the delivery center, one or more examination locations, and the sterilization/cleaning facility, and the delivery time of the device, the examination time, and the like are determined based on an average movement speed of a vehicle as the transport device, a distance of the delivery route, and a traffic state, and thus, efficient scheduling may be performed.

In other words, the medical procedure preparation apparatus set forth in the present example and the like includes at least one processor configured to determine a scheduled performing time of a medical procedure and a scheduled preparation start time (including an intestinal cleansing start time) of the medical procedure, based on the medical procedure performing location information that is information about the location where the medical procedure is performed, and arrival/departure location information that is information about arrival/departure location of a medical device to be used in the medical procedure.

Note that in the description above, the schedule creation unit 41 constructed as a Web application is accessed by the user terminal, for example, but instead, an application including the function of the schedule creation unit 41 may be installed in the user terminal.

### Third Embodiment

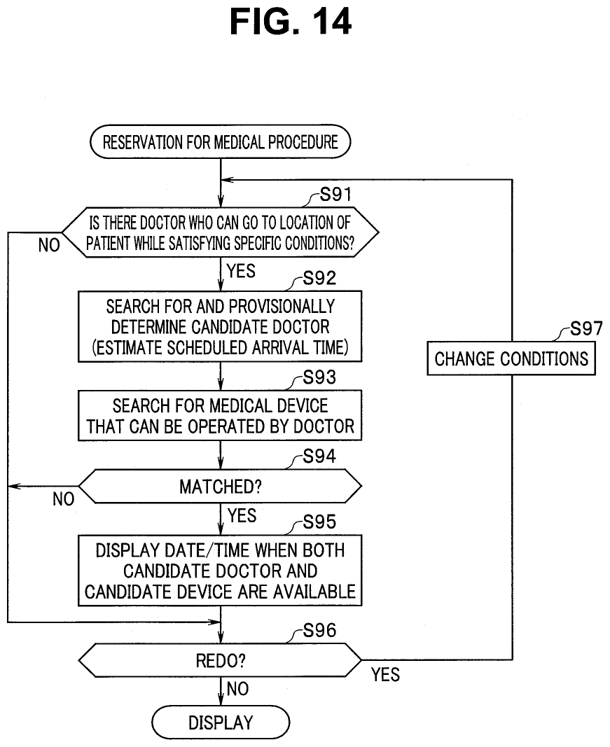

FIG. 14 is a flowchart showing an operation flow adopted in a third embodiment. A hardware configuration of the present embodiment is the same as the hardware configuration in FIG. 1. The first embodiment describes an example where a medical device is carried inside a hospital. The third embodiment is an example where the medical device is carried from the storage location by a car. Furthermore, the present embodiment is an example where the examinee himself/herself makes a reservation for a medical procedure including selection of a doctor.

As described above, examination at home is expected to increase in the future due to a reason such as reduction in the size of medical devices. In such a case, not only movement of the doctor but delivery of the medical device according to the type of the examination must be taken into account.

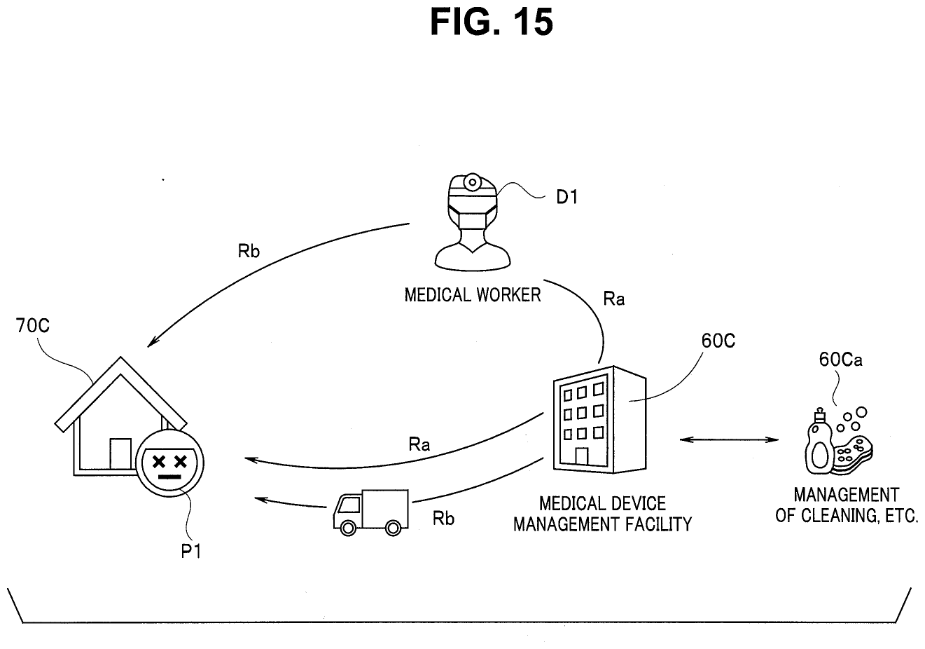

FIG. 15 is an explanatory diagram showing an example of a delivery route of the medical device according to the third embodiment. The example in FIG. 15 shows an example where an examination location 70C that is the medical procedure performing location is home of an examinee (patient) P1. A medical worker D1, such as a doctor or a nurse, goes to the examination location 70C to perform a medical procedure. The medical device is stored in a device storage space 60C in a medical device management facility or the like. Note that the medical device that is stored in the device storage space 60C is already cleaned by a cleaning device 60Ca as necessary.

A route Ra in FIG. 15 indicates that the medical worker D1 goes to the device storage space 60C en route to the examination location 70C to pick up the medical device, and then heads to the examination location 70C. Furthermore, a route Rb indicates that the medical worker D1 heads directly to the examination location 70C, and that the medical device is delivered from the device storage space 60C to the examination location 70C. In other words, the route Rb indicates that the medical worker D1 obtains the medical device at the examination location 70C that is a site of the medical procedure.

In other words, FIG. 15 shows an example where the medical device is not stored at a hospital where the medical worker D1 is stationed, but is stored in the device storage space 60C. In the example in FIG. 15, the medical device needs not to be stored in a hospital, and cost necessary for storing the medical device may be reduced. Furthermore, a latest medical device stored in the device storage space 60C may be used, and quality of a medical service that is provided may be increased.

Nowadays, a user may be able to make a reservation for a medical procedure, such as endoscopy, by accessing a reservation site of a certain hospital using a communication line such as the Internet. In such a case, a reservation for an examination room in the hospital can be made by selecting a vacant time for the examination that is provided by the hospital. In contrast, in the present embodiment, the patient P1 may set the examination location 70C and a schedule for delivery of the medical device with a certain degree of freedom, by, for example, accessing the schedule creation unit 41 constructed as a Web application on a server on a cloud, by using a user terminal such as a smartphone, not shown. Moreover, a doctor can also be selected based on information stored in the doctor information memory 23 in the medical chart information management unit 20.

For example, if the patient P1 selects home as the examination location 70C, because a stranger is to be invited into a private space, the patient P1 may want to select a doctor as the medical worker D1. Moreover, in some cases, the patient P1 may want to select the type of examination. In such a case, the patient P1 inputs specific conditions by operating the user terminal. Specific conditions are the type of treatment/examination, preference of a doctor, specification of date/time, specification of location, and the like. The schedule creation unit 41 determines, according to the specific conditions, whether there is a doctor who can go to the location of the patient P1, such as the home of the patient P1 (S91 in FIG. 14). In other words, how busy a doctor is with daily work and a private schedule of the doctor (such matters are recorded in a scheduler in a mobile terminal of the doctor or in a medical institution where the doctor works, and can be referred to and reflected by the schedule creation unit 41) are taken into account, and schedule and date/time when the medical procedure can be performed are taken into account, and thus, house visit by the doctor may be easily performed. If there is no matching doctor, the schedule creation unit 41 transmits a message requesting re-setting (redo) of conditions to the user terminal of the patient P1, and determines whether there is an instruction for redo (S96). When the schedule creation unit 41 receives a change in the conditions from the patient P1 (YES at S96), the schedule creation unit 41 changes the conditions at S97, and proceeds to S91.

The at least one processor can be further configured to receive reservation conditions from an examinee, search the doctor matching the reservation conditions, search the medical device that the doctor is able to operate. Calculation one of (a) and (b) can be further based on the doctor matching.

If the schedule creation unit 41 determines at S91 that there is a doctor who can go to the home or the like of the patient P1, the schedule creation unit 41 provisionally determines a search result regarding the doctor, and estimates a scheduled time when the doctor (candidate doctor) arrives at the home of the patient P1 (S92). The schedule creation unit 41 also searches for a medical device (candidate device) that can be operated by the candidate doctor (S93). Here, an arrival time of the medical device is also estimated and taken into account. The device may be carried by the doctor, picked up en route from a predetermined storage facility or the like by the doctor, or delivered by another delivery system. Whether the device can be used at location (in the present case, home) and date/time according to the specific conditions specified by the patient P1 is determined based on a distance, a delivery route and the like between a delivery source and the home of the patient P1. At S94, the schedule creation unit 41 determines whether there is a combination of a doctor (candidate doctor) and a medical device (candidate device) that satisfy specific conditions (such as date/time) specified by the patient P1 (whether there is a match). If there is no match, the schedule creation unit 41 transmits a message requesting re-setting (redo) of conditions to the user terminal of the patient P1, and determines whether there is an instruction to redo (S96). In other words, the medical procedure preparation apparatus set forth in the present example and the like includes at least one processor, and can determine a candidate for a scheduled performing time of a medical procedure based on medical procedure performing location information that is information about a location where the medical procedure is performed, storage location information that is information about a storage location of a medical device to be used in the medical procedure, and a schedule of a doctor who performs the medical procedure. Such an element is also included in other examples, but is made more apparent in the present example. Calculation one of (a) and (b) can be further based on the schedule of the doctor who performs the medical procedure.

If the schedule creation unit 41 determines that there is a match at S94, the schedule creation unit 41 transmits, to the user terminal, information about date/time (OK) when both the candidate doctor and the candidate device are able to arrive at the home of the patient P1, and causes the information to be displayed. The patient P1 operates the user terminal, and if the patient P1 wishes to redo, the patient transmits such changes to the schedule creation unit 41. In such a case, the schedule creation unit 41 changes the conditions at S97, and proceeds to S91. Furthermore, if the patient P1 does not wish to redo, the patient transmits no changes to the schedule creation unit 41. The schedule creation unit 41 thus creates a schedule for the medical procedure for the patient P1, and transmits the schedule to each display device to be displayed.

As described above, in the present embodiment, if a patient wishes an examination to be performed at home or the like, the patient himself/herself may make a reservation for the examination. In such a case, the patient may select not only the type of the examination, but also a doctor, and the schedule creation unit may select the doctor and a medical device according to the wish of the user, and create a schedule that takes into account movement time of the doctor and the medical device. Furthermore, the medical device needs not to be kept at the hospital. As a result, cost may be reduced, and quality of the service that is provided may be improved by using a latest medical device.

Note that in the description above, the schedule creation unit 41 constructed as a Web application is accessed by the user terminal, for example, but instead, an application including the function of the schedule creation unit 41 may be installed in the user terminal.

Moreover, in the embodiments described above, the transport device is described by taking a cart or the like as an example, but devices such as a self-propelled robot, a human- or pet-shaped self-propelled device, a cart driven by such a robot, and a drone flying in the air may also be adopted as the transport device. Such a transport device is effective as an important solution for transport of a medical device in relation to healthcare in a large hospital, a remote island, or an isolated place, and has a good track record, and may be adopted as the transport device in each embodiment described above.

Moreover, preparation of apparatus and device has been mainly described in many examples, but some preparations must be made on the part of the examinee, the doctor, and the medical worker, and such preparations and information about time period for such preparations may be recorded together in a knowledge database, and similar guides may be provided. The preparation apparatus 10 in FIG. 1 may include a communication unit configured to wirelessly transmit such information to a mobile terminal of each person. The drawing omits illustration of a wireless communication unit provided in the apparatus or a wired communication unit provided in the apparatus as necessary.

The present disclosure is not limited to the respective embodiments described above, and components may be modified and embodied without departing from the subject matter in the practice of the disclosure. Moreover, various disclosures can be made by combining a plurality of components disclosed in the respective embodiments as appropriate. For example, some components among the components shown in the embodiments may be excluded. Moreover, components of different embodiments may be combined as appropriate.

Moreover, of the technologies described herein, most of the controls and functions described mainly with reference to the flowcharts may be set by programs, and the control and functions described above may be implemented by a computer reading and executing the programs. The programs may be entirely or partially recorded or stored, as a computer program product, in a portable medium such as a non-volatile memory including a flexible disk or a CD-ROM, a hard disk, or a storage medium such as a volatile memory. The computer program product may be distributed or provided at the time of shipping, via a portable medium or a communication line. A user may easily implement the medical procedure preparation apparatus of the present embodiments by downloading the programs via a communication network and installing the programs in a computer, or by installing the programs in a computer from a recording medium.

Example 1. A medical procedure preparation apparatus comprising a processor, wherein
the processor determines a scheduled performing time of a medical procedure or a scheduled preparation start time of a medical device that is used in the medical procedure, based on medical procedure performing location information that is information about a location where the medical procedure is performed, and storage location information that is information about a storage location of the medical device.

Example 2. The medical procedure preparation apparatus according to Example 1, wherein the processor determines a relationship between the scheduled performing time of the medical procedure and the scheduled preparation start time of the medical device, and determines the scheduled performing time of the medical procedure or the scheduled preparation start time of the medical device.

Example 3. The medical procedure preparation apparatus according to Example 2, wherein the processor determines the relationship between the scheduled performing time of the medical procedure and the scheduled preparation start time of the medical device according to a movement speed of a transport device that moves between the medical procedure performing location and the storage location.

Example 4. The medical procedure preparation apparatus according to Example 1, wherein the processor determines the scheduled performing time of the medical procedure or the scheduled preparation start time of the medical device based not only on the medical procedure performing location information and the storage location information, but also on transport device location information that is information about a placement location of the transport device.

Example 5. The medical procedure preparation apparatus according to Example 1, wherein
the processor creates a schedule of the medical procedure including the scheduled performing time of the medical procedure and the scheduled preparation start time of the medical device, based on medical chart information including information about a type of the medical procedure on an examinee, device information including information about a first storage location where a first medical device corresponding to the type of the medical procedure is stored, first medical procedure performing location information that is information about a first medical procedure performing location where the medical procedure is performed on the examinee, first storage location information that is information about a location of the first storage location, first transport device location information that is information about a placement location of a first transport device that carries the medical device from the first storage location to the first medical procedure performing location, and information about a movement speed of the first transport device, and
presents the schedule that is created to at least one of the examinee, a doctor who performs the medical procedure, or a medical worker who carries the medical device.

Example 6. The medical procedure preparation apparatus according to Example 5, wherein the device information includes information about cleaning of the medical device, and information about reception of the medical device.

Example 7. The medical procedure preparation apparatus according to Example 5, wherein the processor performs schedule display to present the schedule of the medical procedure, and includes information about the first medical device in the schedule display.

Example 8. The medical procedure preparation apparatus according to Example 7, further comprising a display device configured to display information about the medical device that is placed on the transport device.

Example 9. An endoscopy preparation apparatus comprising a processor, wherein
the processor determines a cleaning start time of an endoscope apparatus that is used in endoscopy and a scheduled preparation start time for transport of the endoscope apparatus, based on endoscopy performing location information that is information about a location where the endoscopy is performed, and storage location information that is information about a storage location of the endoscope apparatus.

Example 10. A medical procedure preparation apparatus comprising a processor, wherein
the processor
   obtains a first medical procedure performing location and a desired medical procedure performing time point,
   obtains a second medical procedure performing location,
   calculates a transport time of a medical device based on a positional relationship between the first and second medical procedure performing locations, and
   determines a desired medical procedure performing time point at the second medical procedure performing location, based on the desired medical procedure performing time point at the first medical procedure performing location and the transport time that is calculated.

Example 11. A medical procedure preparation apparatus comprising a processor, wherein
the processor
   receives a reservation that is made by a user,
   searches for a doctor matching reservation conditions set by the user,
   searches for a medical device that the doctor who is retrieved is able to operate, and
   performs scheduling associating the doctor and the medical device with a reservation date/time, in a case where the doctor and the medical device that are retrieved satisfy the reservation conditions.

Example 12. A medical procedure preparation method comprising:
obtaining medical procedure performing location information that is information about a location where a medical procedure is performed;
obtaining storage location information that is information about a storage location of a medical device that is used in the medical procedure; and
determining a scheduled performing time of the medical procedure or a scheduled preparation start time of the medical device, based on the medical procedure performing location information and the storage location information.

Example 13. A medical procedure preparation method comprising:
obtaining a first medical procedure performing location and a desired medical procedure performing time point;
obtaining a second medical procedure performing location;
calculating a transport time of a medical device based on a positional relationship between the first and second medical procedure performing locations; and
determining a desired medical procedure performing time point at the second medical procedure performing location, based on the desired medical procedure performing time point at the first medical procedure performing location and the transport time that is calculated.

Example 14. A medical procedure preparation method comprising:
receiving a reservation that is made by a user;
searching for a doctor matching reservation conditions set by the user;
searching for a medical device that the doctor who is retrieved is able to operate; and
performing scheduling associating the doctor and the medical device with a reservation date/time, in a case where the doctor and the medical device that are retrieved satisfy the reservation conditions.

Example 15. A medical system comprising:
a medical procedure preparation apparatus including a processor configured to determine a scheduled performing time of a medical procedure or a scheduled preparation start time of a medical device that is used in the medical procedure, based on medical procedure performing location information that is information about a location where the medical procedure is performed, and storage location information that is information about a storage location of the medical device; and
a display device configured to display information about at least one of the scheduled performing time or the scheduled preparation start time.

Example 16. A non-transitory recording medium recording a medical procedure preparation program, wherein the medical procedure preparation program causes a computer to:
obtain medical procedure performing location information that is information about a location where a medical procedure is performed,
obtain storage location information that is information about a storage location of a medical device that is used in the medical procedure, and
determine a scheduled performing time of the medical procedure or a scheduled preparation start time of the medical device, based on the medical procedure performing location information and the storage location information.

Example 17. A non-transitory recording medium recording a medical procedure preparation program, wherein the medical procedure preparation program causes a computer to:
obtain a first medical procedure performing location and a desired medical procedure performing time point,
obtain a second medical procedure performing location,
calculate a transport time of a medical device based on a positional relationship between the first and second medical procedure performing locations, and
determine a desired medical procedure performing time point at the second medical procedure performing location, based on the desired medical procedure performing time point at the first medical procedure performing location and the transport time that is calculated.

Example 18. A non-transitory recording medium recording a medical procedure preparation program, wherein the medical procedure preparation program causes a computer to:
receive a reservation that is made by a user,
search for a doctor matching reservation conditions set by the user,
search for a medical device that the doctor who is retrieved is able to operate, and
perform scheduling associating the doctor and the medical device with a reservation date/time, in a case where the doctor and the medical device that are retrieved satisfy the reservation conditions.

Example 19. A medical procedure preparation apparatus comprising a processor, wherein
the processor determines a candidate for a scheduled performing time of a medical procedure based on medical procedure performing location information that is information about a location where the medical procedure is performed, storage location information that is information about a storage location of a medical device that is used in the medical procedure, and a schedule of a doctor who performs the medical procedure.

Example 20. A medical procedure preparation apparatus comprising a processor, wherein
the processor determines a scheduled performing time of a medical procedure and a scheduled preparation start time of the medical procedure, based on medical procedure performing location information that is information about a location where the medical procedure is performed, and arrival/departure location information that is information about arrival/departure location of a medical device that is used in the medical procedure.

What is claimed is:

1. A medical procedure preparation system, comprising:
an input interface;
a display; and
at least one processor comprising hardware, the at least one processor configured to:
receive an input containing (i) a medical procedure location where a medical procedure is performed, and (ii) a storage location where a medical device is stored;
calculate (iii) a distance between (i) the medical procedure location and (ii) the storage location;
calculate one of (a) a preparation start time of the medical device used in the medical procedure and (b) a performing time of the medical procedure using the medical device; and
output a display image indicating one of (a) and (b) to the display,
wherein calculation one of (a) and (b) is based on (iii) the distance and (iv) a movement speed of a transport device.

2. The medical procedure preparation system according to claim 1, wherein the at least one processor is further configured to:
receive a medical chart information;
determine the medical device used in the medical procedure based on the medical procedure; and
wherein the medical chart information includes a type of the medical procedure to be performed on an examinee.

3. The medical procedure preparation system according to claim 1, wherein the display is configured to display the display image.

4. The medical procedure preparation system according to claim 1, wherein calculation one of (a) and (b) is further based on (vi) a location of the transport device that moves between (i) the medical procedure location and (ii) the storage location.

5. The medical procedure preparation system according to claim 1, wherein the at least one processor is further configured to:
generate a schedule of the medical procedure including (a) the preparation start time and (b) the performing time;
wherein generating the schedule is based on one or more of (i) the medical procedure location, (ii) the storage location, (iv) the movement speed, (v) a location of the transport device, and (vi) a medical chart information,
wherein (v) the location of the transport device that carries the medical device from (ii) the storage location to (i) the medical procedure location,
wherein (vi) the medical chart information includes a type of the medical procedure to be performed on an examinee, and
output the schedule to at least one of the examinee or a medical professional.

6. The medical procedure preparation system according to claim 5, wherein the display image comprises a first display image, and
wherein the at least one processor is further configured to:
output a second display image indicating the medical device that is already prepared and placed on the transport device.

7. The medical procedure preparation system according to claim 6, wherein the at least one processor is further configured to:
switch the first display image and the second display image.

8. The medical procedure preparation system according to claim 1, wherein the at least one processor is further configured to:
calculate one of (c) an updated preparation start time of the medical device and (d) an updated performing time of the medical procedure, and
wherein calculation one of (c) and (d) is based on (iii) the distance, (iv) the movement speed of the transport device, (v) a preparation status that is already prepared and placed on the transport device and (vi) a location of the transport device that moves between (i) the medical procedure location and (ii) the storage location.

9. The medical procedure preparation system according to claim 1, wherein the display image further indicates (i) the medical procedure location and (ii) the storage location.

10. The medical procedure preparation system according to claim 1, wherein calculation one of (a) and (b) is further based on (v) a cleaning status of the medical device.

11. The medical procedure preparation system according to claim 1, wherein calculation one of (a) and (b) is further based on a time of arrival of the medical device at (i) the medical procedure location.

12. The medical procedure preparation system according to claim 1, wherein the medical device comprises an endoscope.

13. The medical procedure preparation system according to claim 1, wherein
when the at least one processor is configured to calculate (a), calculation of (a) is further based on (b), and
when the at least one processor is configured to calculate (b), calculation of (b) is further based on (a).

14. The medical procedure preparation system according to claim 1, wherein the at least one processor is further configured to:
receive reservation conditions from an examinee;
search a doctor matching the reservation conditions;
search the medical device that the doctor is able to operate, and
wherein calculation one of (a) and (b) is further based on the doctor matching.

15. The medical procedure preparation system according to claim 1, wherein calculation one of (a) and (b) is further based on a schedule of a doctor who performs the medical procedure.

16. The medical procedure preparation apparatus according to claim 1, wherein calculation one of (a) and (b) is further based on arrival/departure location information indicating an arrival/departure location of the medical device between (i) the medical procedure location and (ii) the storage location.

17. The medical procedure preparation system according to claim 1, further comprising:
the transport device configured to the medical device from (ii) the storage location to (i) the medical procedure location.

18. The medical procedure preparation system according to claim 8, wherein the display image comprises a first display image, and
wherein the at least one processor is further configured to:
output a second display image indicating (c) the updated preparation start time to the display; and
switch the first display image and the second display image.

19. A medical procedure preparation method, comprising:
receiving an input containing (i) a medical procedure location where a medical procedure is performed, and (ii) a storage location where a medical device is stored from an input interface;
calculating (iii) a distance between (i) the medical procedure location and (ii) the storage location where the medical device is stored;
calculating one of (a) a preparation start time of the medical device used in the medical procedure and (b) a performing time of the medical procedure using the medical device; and
outputting a display image indicating one of (a) and (b) to a display, and
wherein calculation one of (a) and (b) is based on (iii) the distance and (iv) a movement speed of a transport device.

20. A medical procedure preparation method, comprising:
receiving an input signal from an input interface;
obtaining, based on the input signal:
a medical procedure location where the medical procedure is performed;
a storage location where a medical device is stored, wherein the medical device is used in the medical procedure; and
a performing time of the medical procedure using the medical device;
calculating a preparation start time of the medical device; and
outputting the preparation start time.
